(19) United States
(12) Patent Application Publication
(10) Pub. No.: US 2021/0228771 A1
(43) Pub. Date: Jul. 29, 2021

(54) BIOLOGIC MATERIAL COMPRISING A CROSSLINKED STRUCTURAL PROTEIN AND MACROPHAGES SEEDED ON THE CROSSLINKED STRUCTURAL PROTEIN

(71) Applicant: CASE WESTERN RESERVE UNIVERSITY, Cleveland, OH (US)

(72) Inventors: Ozan AKKUS, Cleveland Heights, OH (US); Adonis HIJAZ, Westlake, OH (US)

(21) Appl. No.: 17/158,424

(22) Filed: Jan. 26, 2021

Related U.S. Application Data
(60) Provisional application No. 62/966,308, filed on Jan. 27, 2020.
(60) Provisional application No. 63/066,863, filed on Aug. 18, 2020.

(51) Int. Cl. A61L 27/22; A61L 27/24; A61L 27/36; A61L 27/52

(52) U.S. Cl. CPC A61L 27/227; A61L 27/24; A61L 27/225; A61L 2430/40; A61L 27/3683; A61L 27/52; A61L 27/222

(57) ABSTRACT

A biologic material is disclosed. The biologic material comprises a crosslinked structural protein and macrophages seeded on the crosslinked structural protein. A method of use of the biologic material for an immunoregenerative treatment in a patient in need thereof also is disclosed. The method comprises steps of: (1) seeding the macrophages on the crosslinked structural protein, thereby obtaining the biologic material; and (2) implanting the biologic material into the patient.

### STATEMENT OF GOVERNMENT-SPONSORED RESEARCH

This invention was made with government support under AR063701 awarded by the National Institutes of Health. The government has certain rights in the invention.

### FIELD OF THE INVENTION

The present invention relates generally to biologic materials, and more particularly to biologic materials comprising a crosslinked structural protein and macrophages seeded on the crosslinked structural protein.

### BACKGROUND OF THE INVENTION

Biomaterials are used in a wide range of medical applications to improve or replace a natural function. Biomaterials are materials, whether natural, synthetic, or a combination, that interact with biological systems. Biomaterials can serve, for example, as scaffolds for tissue regeneration. Medical applications in which biomaterials are used include treatment of stress urinary incontinence (also termed SUI), treatment of pelvic organ prolapse (also termed POP), hernia repair, and orthopedic applications.

Considering stress urinary incontinence in detail, SUI is involuntary leakage of urine due to weakening of pelvic muscles and ligaments which constrict the urethra. SUI occurs predominantly in the female population because of pregnancy and parity related trauma to the pelvic region (11-13). One in three women above the age 60 experience SUI (14). SUI is associated with a cost of tens of billions of dollars (15).

A mid-urethral sling is a tape-shaped biomaterial that supports and constricts the urethra to maintain continence. It is estimated that 750,000 sling procedures/year occur in the United States (2). By 2020, the number of sling procedures in the U.S. is projected to be 1,000,000/year (16). Polypropylene meshes and fascial autografts are used as slings.

A number of polymers have been used in the past and polypropylene dominates the sling field (17-19). Inflammatory response to polypropylene slings (also termed PS) results in fibrosis which reinforces the support by the sling and it is one of the reasons why polypropylene slings have been successful in providing long term continence (20-22). On the other hand, polypropylene slings may induce vaginal atrophy and in some cases vaginal erosion. Erosions are the second most frequently reported adverse events to FDA for SUI procedures (3, 4, 23). Erosions are speculated to be caused by a myriad of factors such as degradative enzymes released from the permanent foreign body response or the stress shielding associated with stiff healing response (3, 5). The literature reports erosions variably between 2.5%-7.3% (reviewed in 24), including FDA's Ob&Gyn Advisory committee's estimate of 5% of all procedures (23). At an estimated 750,000+ surgeries/year, 35,000+ erosions/year is significant. Other drawbacks associated with polypropylene slings are: a) polypropylene slings may be contraindicated for atrophied vagina and in revision of failed polypropylene slings, b) some patients opt out of polypropylene slings due to negative publicity on polypropylene meshes used in pelvic prolapse procedures, and c) voiding dysfunction and painful intercourse due to mesh related foreign body sensation (3, 5). In these situations, autograft sling becomes the treatment of choice for a non-negligible sub-population of SUI patients.

Fascial autograft treatment results in excellent clinical outcome in providing long term continence (6-8, 25), reaffirmed by a randomized clinical trial (26). Unlike polypropylene slings, erosion is not encountered in fascial autograft-based repair. Major downsides of fascial autografts are: donor site morbidity (e.g. abdominal hernia (27)) and prolonging the surgery and the recovery (28). Fascial autograft procedures requires hospital stay, increasing the cost of treatment.

Xenografts or allografts, such as decellularized dermis or subintestinal mucosa, were used clinically in the past but they are largely abandoned at the present. Most xenografts lack a connected microporous network which limits host integration and some result in immune rejection (20, 21, 29, 30). Physical properties of xenografts degrade prematurely without being replaced by a robust connective tissue that would serve the support function (27, 31), resulting in rejection or recurrence of incontinence.

A biologic material that remodels into vascularized native tissue would improve the treatment of tens of thousands of patients per year with respect to treatment of SUI. Such a biologic material also may improve treatment of additional patients with respect to POP, hernia, orthopedic applications, and potentially additional medical applications.

Macrophages are central to the orchestration of repair and regeneration of healing tissues (32, 33). Macrophages are large mononuclear phagocytic cells that serve as scavenger cells, pathogen recognition cells and a source of proinflammatory cytokines in innate immunity, antigen presenting cells, and effector phagocytic cells in humoral and cell-mediated immunity. Macrophages are migratory cells that are derived from bone marrow. Macrophages are found in most tissues of the body. Macrophages polarize mainly into M1 and M2 types which are proinflammatory and anti-inflammatory, respectively. M1 phenotype is predominant at the onset of wound healing site to mitigate infection and phagocytosis of damaged cells. M2 polarization emerges during latent repair phase and M2 macrophages express a variety of trophic factors which promote vascularization (34-37), matrix synthesis (38) and chemotactic attraction of stem cells and myofibroblasts to the wound site (39). M2 macrophages produce a variety of factors which are stimulatory to matrix formation such as PDGF-BB, FGF2 and IGF1 (34, 40). Importantly, isolation of monocytes from blood and their induction to M2-macrophages is well established in the field (4144). Therefore, macrophages are accessible cells and their collection is facile for therapeutic purposes.

Recent review papers (45, 46) proposed the use of macrophages for regenerative purposes either by way of modifying biomaterials to attract host macrophages, or by direct delivery of macrophages. Direct delivery ascertains that desired cell types are delivered at sufficient levels to the host. However, attempts to realize macrophage driven immunoregenerative studies are highly limited in the literature. The limited examples include a study by Spiller et al. (43), which involved delivery of IFN gamma and IL4 sequentially from demineralized trabecular bone to attract M1 and M2 macrophages, respectively, from the host, and a study by Rybalko et al. (47), in which M1 macrophages were administered to ischemic muscles.

A need exists for improved biologic materials that can remodel into vascularized native tissue for improved treatment of SUI and other medical applications.

### BRIEF SUMMARY OF THE INVENTION

A biologic material is provided. The biologic material comprises (a) a crosslinked structural protein; and (b) macrophages seeded on the crosslinked structural protein.

In some embodiments, the crosslinked structural protein comprises one or more of a crosslinked collagen, a crosslinked gelatin, a crosslinked elastin, or a crosslinked keratin. In some of these embodiments, the crosslinked structural protein comprises a crosslinked collagen.

In some embodiments, the crosslinked structural protein has been crosslinked with an iridoid crosslinking agent. In some of these embodiments, the crosslinked structural protein has been crosslinked with one or more of genipin, loganin aglycone, oleuropein aglycone, or E-6-O-methoxycinnamoyl scandoside methyl ester aglycone.

In some embodiments, the crosslinked structural-protein forms a crosslinked structural-protein mesh. In some of these embodiments, the crosslinked structural-protein mesh comprises a crosslinked collagen mesh. In some of these embodiments, the crosslinked collagen mesh comprises a crosslinked woven collagen mesh.

In some embodiments, the crosslinked structural-protein forms a crosslinked structural-protein gel. In some of these embodiments, the crosslinked structural-protein gel comprises a crosslinked collagen gel.

In some embodiments, the macrophages comprise autologous-blood derived macrophages and/or allogeneic-blood derived macrophages.

In some embodiments, the macrophages comprise M2 macrophages.

In some embodiments, the macrophages have been seeded on the crosslinked structural protein at a seeding density of 5,000 to 100,000 macrophages/mm³ of the biologic material.

In some embodiments, to the extent that human or animal cells other than macrophages are present on the crosslinked structural protein of the biologic material, more macrophages are present than the other human or animal cells.

In some embodiments, to the extent that human or animal cells other than macrophages are attached to the crosslinked structural protein of the biologic material, more macrophages are attached than the other human or animal cells.

A method of use of the biologic material for an immunoregenerative treatment in a patient in need thereof also is provided. The method comprises steps of: (1) seeding the macrophages on the crosslinked structural protein, thereby obtaining the biologic material; and (2) implanting the biologic material into the patient.

In some embodiments, the macrophages comprise M2 macrophages derived from monocytes, the method further comprising steps of: (0.1) treating the monocytes with Macrophage Colony Stimulating Factor to obtain M0 macrophages; and (0.2) treating the M0 macrophages with a mixture of TGF-β, IL-4, IL-10, and/or IL-13 to obtain the M2 macrophages.

In some embodiments, the macrophages comprise M2 macrophages derived from M0 macrophages, and the method further comprises a step (0.3) of treating the M0 macrophages with genipin in a solution to obtain the M2 macrophages.

In some embodiments, the method further comprises treating the macrophages with IL-4 during step (1).

In some embodiments, the immunoregenerative treatment comprises one or more of treatment of stress urinary incontinence, treatment of pelvic organ prolapse, hernia repair, or an orthopedic application.

Another biologic material also is provided. The biologic material comprises (a) a crosslinked amine-functionalized biodegradable polymer; and (b) macrophages seeded on the crosslinked amine-functionalized biodegradable polymer.

In some embodiments, the crosslinked amine-functionalized biodegradable polymer comprises one or more of a crosslinked amine-functionalized biodegradable polyester, a crosslinked amine-functionalized poly lactic-co-glycolic acid, a crosslinked amine-functionalized polycaprolactone, a crosslinked amine-functionalized biodegradable polysaccharide, or a crosslinked amine-functionalized chitosan.

In some embodiments, the crosslinked amine-functionalized biodegradable polymer has been crosslinked with an iridoid crosslinking agent. In some of these embodiments, the crosslinked amine-functionalized biodegradable polymer has been crosslinked with one or more of genipin, loganin aglycone, oleuropein aglycone, or E-6-O-methoxycinnamoyl scandoside methyl ester aglycone.

In some embodiments, the crosslinked amine-functionalized biodegradable polymer forms a crosslinked amine-functionalized biodegradable polymer mesh.

In some embodiments, the crosslinked amine-functionalized biodegradable polymer forms a crosslinked amine-functionalized biodegradable polymer gel.

In some embodiments, the macrophages comprise autologous-blood derived macrophages and/or allogeneic-blood derived macrophages.

In some embodiments, the macrophages comprise M2 macrophages.

In some embodiments, the macrophages have been seeded on the crosslinked amine-functionalized biodegradable polymer at a seeding density of 5,000 to 100,000 macrophages/mm³ of the biologic material.

In some embodiments, to the extent that human or animal cells other than macrophages are present on the crosslinked amine-functionalized biodegradable polymer of the biologic material, more macrophages are present than the other human or animal cells.

In some embodiments, to the extent that human or animal cells other than macrophages are attached to the crosslinked amine-functionalized biodegradable polymer of the biologic material, more macrophages are attached than the other human or animal cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. (A) Collagen threads. (B) A woven CollaMesh article. (C) Implantation of a woven collagen article holding sutures as a mid-urethral sling in rat. Fabrication and characteristics of threads and woven articles have been described (1). Arrowheads highlight in-plane cylindrical pores.

FIG. 2. (A) Genipin crosslinked collagen and (B) EDC/NHS crosslinked collagen. Genipin crosslinking of collagen resulted in greater amount of collagen deposition (arrowheads) than EDC/NHS crosslinked collagen as manifested by extensive presence of blue stained fibers in Masson's trichome stained sections. “*” denotes cross-lined collagen threads. (C) Genipin crosslinked meshes also had a greater amount of CD206+M2-macrophages than that on EDC/NHS crosslinked meshes at 5 months. (D) Mousa RAW 264.7 macrophages adhere and establish focal adhesions on genipin crosslinked collagen (f-actin staining, 24 hours post seeding).

FIG. 3. Average modulus of meshes obtained from tensile tests indicate that genipin cross-linked meshes eventually converge to a stiffness that matches that of vaginal tissue whereas EDS cross-linked meshes lose mechanical robustness at 5 months. Squares indicate a value is significantly different from the preceding value (intra-group comparison) and stars indicate that a given value is significantly different from native tissue at that time point (inter-group comparison). Dashed lines highlight mean values for native tissues.

FIG. 4. (A) A knitted collagen sling is shown. The inset in “A” shows a macrograph of collagen yarns with a loop size of 0.75 mm. (B) An in-house custom made knitting machine for making knitted collagen slings is shown. (C) Placement of a sling in a ewe is shown. D) Sling is tensioned at the abdomen to secure in place.

FIG. 5. Schematic of isoelectric focusing.

FIG. 6. Isolation and growth of rat bone derived macrophages.

FIG. 7. A) Demonstration of midline incision. B) Dropping of bones with muscle attached into DPBS. C) Flushing bone marrow using 23 G needle.

FIG. 8. A) Genipin cross-linked meshes. B) Sterilized collagen scaffolds. C) Cell seeding on scaffolds.

FIG. 9. Morphotypes of polarized bone marrow derived (A) M0, (B) M1, and (C) M2 macrophages, shown at (left) 5× magnification and (right) 25× magnification.

FIG. 10. Flow Jo analyzer for titration of antibodies.

FIG. 11. Characteristics of (A) M0, (B) M1, and (C) M2 macrophages determined by flow cytometry. Representative images of (left) CD68, (middle) CD86, and (right) CD163 expression in the macrophages.

FIG. 12. Quantitative analysis of (A) CD68, (B) CD86, and (C) CD163 positive cells. Data are shown as mean±standard deviation (n=4).

FIG. 13. Light microscopy (up) and zoomed-in (down) images of genipin crosslinked collagen mesh. Scale bar is 10 mm.

FIG. 14. Histograms representing percentage adhesion versus time for (left) M0, (middle) M1, and (right) M2 macrophages, at 2 hours, 4 hours, and 6 hours for each, on genipin crosslinked collagen scaffolds.

FIG. 15. Representation of cell proliferation as percentage of Alamar blue dye reduction at (left) day 1, (middle) day 2, and (right) day 3, for M0, M1, and M2 macrophages on genipin crosslinked collagen scaffolds.

FIG. 16. Representative images of (left) DAPI staining, (middle) phalloidin staining, and (right) DAPI and phalloidin staining of M0, M1, and M2 macrophages on genipin crosslinked collagen scaffolds.

FIG. 17. Characteristics of M0, M1, and M2 macrophages determined by immunocytochemistry.

FIG. 18. Western blot data for macrophages seeded on genipin crosslinked mesh after 72 hours.

FIG. 19. Western blot data for macrophages seeded on uncrosslinked mesh and treated with genipin after 72 hours.

FIG. 20. Fabrication of filament wound collagen scaffolds. (A) Electrochemical compaction of type 1 collagen solution. (B) Collection of collagen threads in isopropanol jar. (C) Demonstration of single electrochemical aligned collagen (ELAC) thread. Scale bar is 0.7 mm. (D) Collection of collagen yarns in continuous lengths onto spool. (E) Computer numeric controlled (CNC) filament winding of collagen yarns as scaffolds. (F) Recovery of collagen scaffold from the mandrel. (G) Rectangular-shaped uncrosslinked collagen scaffold. Scale bar is 10 mm. (H) Light microscopy image of rectangular-shaped genipin crosslinked collagen scaffold. Scale bar is 10 mm.

FIGS. 21A-E. Characteristics of M0, M1, and M2 macrophages determined by Flow Cytometry (FCM). FIG. 21A and FIG. 21B show representative FCM pictures of CD68 (FITC), CD86 (PE) and CD163 (AlexaFluor 647) expression in M0, M1 and M2 macrophages. The antibodies were gated in cyan color in the corresponding cell population. Black lines in FIG. 21B show matched isotype control staining. FIGS. 21C-E show quantitative analysis of CD68+, CD86+, CD163+ positive cells. Data represents mean±standard deviation of a total of five independent (n=5). Significance was determined using Mann-Whitney test. *—to indicate p<0.05, ‘ns’ to indicate non-significance. Error bars represent standard deviation.

FIG. 22. Characteristics of M0, M1 and M2 macrophages determined by Immunofluorescent Staining. (A) Representative immunofluorescent images of CD68 (green), iNOS (red), and Arginase 1 (orange-red) expression in M0, M1, and M2 macrophages. Scale bar is 80 um. (B) The immunofluorescent detection of the cytoskeletal structure of macrophage subsets (M0, M1, M2) using Alexa 488-conjugated. Scale bar is 50 μm. Rat bone marrow derived monocytes differentiated into M1 and M2 macrophages using recombinant proteins. Characteristically images of M0 (round), M1 (egg-shape), and M2 (spindle-shape) morphology under microscope. Scale bar is 50 μm. (C) Quantification of cell area of macrophage subsets. (D) Quantification of elongation factor of macrophages using Image J software analysis. Data represent mean±standard deviation of a total of five independent (n=5). Significance was determined using Mann-Whitney test. *—to indicate p<0.05, ‘ns’ to indicate non-significance. Error bars represent standard deviation.

FIG. 23. Demonstration of cytoskeleton structure of M0, M1 and M2 macrophages on genipin crosslinked collagen scaffolds by Immunofluorescent Staining. (A) Representative images of macrophage subtypes seeded on genipin crosslinked collagen scaffolds. M0 and M2 macrophages demonstrated a significant degree of alignment on the surface of the collagen fibers while alignment of M1 cytoskeletal elements was not as readily apparent. (B) Cell area and (C) elongation factor results are shown. Cell shape parameter analysis using image J software. Scale bar is 50 μm. (D) Cell proliferation was determined by Alamar Blue Assay on day 1, 2 and 3. Alamar blue assay results show the proliferation of M0, M1 and M2 cells growing on the surface of genipin crosslinked scaffolds. Errors bars represent the average absorbance considered from four replicate specimens (±SD). GES-genipin crosslinked aligned scaffold. Significance was determined using Mann-Whitney test. *—to indicate p<0.05, ‘ns’ to indicate non-significance. Error bars represent standard deviation.

FIG. 24. Determination of the effects of genipin crosslinked scaffolds on polarization status of M0, M1 and M2 macrophages by Western Blot analysis. (A) Total cell lysates were prepared from M0, M1, M2 macrophages seeded on genipin crosslinked scaffolds. Total cell lysates were prepared using SDS-PAGE, lysates proteins were resolved and then subjected to western blot analysis for the evaluation of arginasel (M2 marker) and iNOS (M1 marker) protein expression. (B) iNOS (M1 marker) protein expression and (C) arginasel (M2 marker) are shown. For protein band density, densitometric analysis was performed and protein expression levels were correlated to express relative control. GES-genipin crosslinked aligned scaffolds. Data represents mean±standard deviation of a total of three independent (n=3). Significance was determined using Mann-Whitney test. *—to indicate p<0.05, ‘ns’ to indicate non-significance. Error bars represent standard deviation.

FIG. 25. Determination of the effects of genipin itself and electrochemical aligned collagen (ELAC) induced cellular elongation on M0 macrophages by Western Blot analysis. (A) Total cell lysates were prepared from M0 macrophages treated with genipin and M0 macrophages seeded on uncrosslinked scaffolds (for cellular elongation). Total cell lysates were prepared using SDS-PAGE, lysates proteins were resolved and then subjected to western blot analysis for the evaluation of arginase 1 (M2 marker) protein expression. (B) For protein band density, densitometric analysis was performed and protein expression levels were correlated to express relative control. Data represents mean±standard deviation of a total of three independent (n=3). Significance was determined using Mann-Whitney test. *—to indicate p<0.05, ‘ns’ to indicate non-significance. Error bars represent standard deviation. GES-genipin crosslinked aligned scaffold, GE—genipin treatment, U-Uncrosslinked scaffold.

FIG. 26. Investigation of signaling pathways involved in M2 polarization by genipin. (A) Total cell lysates were prepared from genipin treated M0 macrophages. Total cell lysates were prepared using SDS-PAGE, lysates proteins were resolved and then subjected to western blot analysis for the evaluation of p-STAT6 and PPAR-gamma expression. (B) PPAR-gamma expression and (C) p-STAT6 expression are shown. For protein band density, densitometric analysis was performed and protein expression levels were correlated to express relative control. Data represents mean±standard deviation of a total of three independent (n=3). Significance was determined using Mann-Whitney test. *—to indicate p<0.05, ‘ns’ to indicate non-significance. Error bars represent standard deviation. GE—genipin treatment.

FIG. 27. (A) M0 macrophages, (B) M1 macrophages, and (C) M2 macrophages are shown. Polarization status of M1 and M2 cells are maintained when seeded on collagen threads. M0 cells (A) and M2 cells (C) elongate along the longer axis of collagen threads whereas M1 cells (B) stay round on collagen threads. (D) Western blots and group definitions are shown. (E) M1 marker iNOS and (F) M2 marker Arg1 are still present after being seeded on collagen threads, whereas M0 macrophages polarize to M2 upon seeding. In (E) and (F) bars are absent for other groups because expressions were absent.

FIG. 28. In vivo response to macrophage seeded genipin crosslinked collagen textiles (GES) are provided. (A) Scaffold and associated soft tissue recovered at 3 months are shown. (B) Modulus of M2 supplemented scaffolds (GES-M2) was the highest. (C) GES-M2 had the highest cellularity. (D) GES-M2 also displayed the myofibroblast marker alpha-SMA most prominently. (E) GES-M2 resulted in the highest amount of de novo collagen and the lowest thickness of fibrous encapsulation.

### DETAILED DESCRIPTION OF THE INVENTION

A biologic material is provided. The biologic material comprises (a) a crosslinked structural protein; and (b) macrophages seeded on the crosslinked structural protein.

Preliminary in vivo data indicate that a biologic material comprising a genipin crosslinked woven collagen mesh (CollaMesh) promotes de novo collagen deposition in amounts comparable to that attained by polypropylene slings at 5 months (9, 10). This success was defined by genipin crosslinker, which is an iridoid crosslinking agent, because crosslinked woven collagen meshes made using conventional crosslinking agents 1-ethyl-3-(3-dimethylaminopropyl)carbodiimide hydrochloride (also termed EDC)/N-hydroxysulfosuccinimide (also termed NHS) did not result in robust healing. The stiffness of healing genipin mesh at 5 months matched that of vaginal tissue (10). Yet, the successful outcome transpired at 5 months. Thus, regeneration rate in collagen meshes can be improved.

Notably, favorable outcome from genipin crosslinked CollaMesh was accompanied by a significant elevation of anti-inflammatory M2-macrophages which are known to promote repair and regeneration through secretion of pro-angiogenic and pro-fibrogenesis factors (10).

Without wishing to be bound by theory, it is believed that delivery of autologous-blood derived M2 macrophages and/or allogeneic-blood derived M2 macrophages via genipin crosslinked collagen meshes will result in faster regeneration; specifically, earlier deposition of collagen, earlier inception of neovascularization and earlier attainment of mechanical robustness than the collagen meshes which are implanted without M2 macrophages. It appears that genipin induces polarization of macrophages to the regenerative M2 type. Furthermore, it is believed that the indicated performance metrics of the corresponding genipin crosslinked collagen meshes to which autologous-blood derived M2 macrophages and/or allogeneic-blood derived M2 macrophages have been delivered will match those of polypropylene slings as the clinical control group. To the best of our knowledge, this is the first time an immunoregenerative approach is used via accessible blood-derived autologous and/or allogeneic macrophages.

As noted, a biologic material is provided. A biologic material is a material including a component, typically a complex macromolecule such as a protein, that has been manufactured in a living organism.

The biologic material can have a material structure corresponding to a scaffold, such as a mesh, or a viscous fluid, such as a gel, or a form intermediate between a scaffold and a viscous fluid, such as a spongy form, among other material structures.

Also as noted, the biologic material comprises a crosslinked structural protein. A structural protein is a protein that possesses a characteristic amino acid sequence, also termed a motif, that repeats and contributes to the mechanical properties of a living organism, cell, or material (111). Exemplary structural proteins include collagen, gelatin, elastin, and keratin.

Collagen refers to a long, fibrous structural protein that is a major component of the extracellular matrix, which provides support to tissues and structure to cells. Collagen includes naturally occurring collagens, such as type I, type II, type III, and type IV collagens, and engineered collagens. Collagen includes collagen in forms of any type, including single-stranded and multi-stranded collagenous proteins or polypeptides, the tropocollagen helix comprised of three polypeptide strands, such as type I collagen, and denatured-collagen products, including gelatin as discussed below, that substantially retain their native or engineered primary amino acid sequence.

Collagen also includes electrochemically aligned collagen (also termed ELAC) (1, 51, 52). Electrochemically aligned collagen is a form of collagen that is suitable for use in research on tissue engineering, biofabrication processes, and generation of biomaterials. Electrochemically aligned collagen corresponds to a solid phase made by transforming solutions of monomeric collagen by electrochemical gradients induced by electrodes. Electrical currents are used to create a varying pH distribution, resulting in rapid compaction of collagen molecules. Mechanical properties of the electrochemically aligned collagen are 10 to 100-fold stronger than conventional forms of collagen. Devices and methods for producing electrochemically aligned collagen are described in U.S. Pat. No. 10,017,868, U.S. Pub. No. 2018/0312988, and PCT publication WO 2018/195409, each of which are incorporated by reference herein in their entirety. Electrochemically aligned collagen can be made as filaments, among other forms. The filaments can be made as threads including single filaments and having extended lengths, e.g. 50 meters, among other forms and lengths. Due to their stronger mechanical properties, electrochemically aligned collagen can be used to make devices, such as implantable slings, that have sufficient mechanical properties to hold sutures and bear mechanical loads (1, 51, 52).

Collagen can be derived from any of various naturally-occurring sources, including humans and animals, and can be isolated and prepared according to conventional methods. Collagen can also be prepared or engineered synthetically based on amino acid and nucleic acid sequences for any of the various collagen types using conventional methods of molecular biology and protein expression. Denatured-collagen products can be produced, for example, through partial hydrolysis of native or engineered fibrous collagen proteins. Threads of electrochemically aligned collagen can be fabricated in continuous length and woven to form devices such as implantable slings (52).

Suitable collagen includes, for example, type I collagen, type II collagen, type III collagen, type IV collagen, other native collagens, synthetic or engineered forms or types of collagen, highly purified recombinant collagen, collagen that is a component of a tissue extract, such as gelatin, and collagen that is a product of denaturation and hydrolysis of a naturally occurring, synthetic, or engineered collagen that substantially retains its primary amino-acid sequence, again such as gelatin. Regarding type I collagen in particular, suitable ranges of average molecular weight include, for example, 60,000-120,000 Daltons. Suitable collagens also include electrochemically aligned collagen.

Gelatin refers to a partially hydrolyzed form of collagen. Gelatin is a heterogeneous mixture of water-soluble proteins of high average molecular weights, present in collagen, the proteins having been extracted from any one of various types of animals by boiling skin, tendon, ligaments, bones, and other organs in water. Gelatin is commercially available in various types, including type A, which is acid-cured gelatin, and type B, which is lime-cured gelatin. Gelatin can be produced from various animals, including pig, cow, and fish. Gelatin can be produced in various ranges of average molecular weights, including 20,000-25,000, 40,000-50,000, and 50,000-100,000 Daltons. Gelatin also can be produced having various Bloom values, including Bloom values of 100 to 300, 200 to 300, or 275 to 300. A suitable gelatin can be any of the various gelatins commercially available from Sigma-Aldrich Inc. (St. Louis, Mo.).

Elastin refers to a structural protein of extracellular matrix that is rich in glycine, valine, alanine, and proline, that is present in connective, vascular, and load-bearing tissues and that has elastic mechanical properties. Elastin is poorly soluble in aqueous solutions. Elastin can be used as an additive for biomaterials including collagen. Elastin can be used to impart significant elasticity on biomaterials.

Keratin refers to a structural protein that is the main protein component of hair, wool, feathers, nails, and horns. Keratin is rich in cysteine, which corresponds to 7 to 20% of the total amino acid residues of the protein. Keratin includes α-keratin, which has an alpha-helical coiled coil structure, and (3-keratin, which has a twisted beta sheet structure. Keratin can include disulfide bonds, for example in wool. Reduction of disulfide bonds of keratin converts the keratin to a highly soluble form. Keratin can be used to prepare insoluble films and biodegradable sponge scaffolds, among other materials.

As noted, the structural protein is a crosslinked structural protein. A crosslink is a bond that links one macromolecule to another. A crosslink can be covalent or ionic. Structural proteins can be crosslinked at functional groups of side chains of their amino acid residues, including the amino group of the side chain of lysine residues, the carboxyl groups of the side chains of aspartate and glutamate residues, and the thiol group of the side chain of cysteine.

Accordingly, in some embodiments, the crosslinked structural protein comprises one or more of a crosslinked collagen, a crosslinked gelatin, a crosslinked elastin, or a crosslinked keratin. In some embodiments, the crosslinked structural protein comprises a crosslinked collagen. The crosslinked collagen can be, for example, a crosslinked electrochemically aligned collagen.

Suitable crosslinking agents include iridoid crosslinking agents, such as genipin, loganin aglycone, oleuropein aglycone, and E-6-O-methoxycinnamoyl scandoside methyl ester aglycone. Iridoids are compounds that occur naturally in plants (112). Iridoids have a characteristic structure including a hexa-ring with an alkene-ether bond (112). More than 1400 plant iridoids have been isolated and identified (112). Iridoid crosslinking agents can be made from iridoids, for example by hydrolyzing iridoid glycosides to form their corresponding iridoid aglycones. Without wishing to be bound by theory, it is believed that the use of an iridoid crosslinking agent, instead of conventional crosslinking agents such as EDC/NHS, for crosslinking the structural protein to obtain the biologic material provides an advantage of improved healing upon use of the biologic material as an implant. As noted above, preliminary in vivo data indicate that a biologic material comprising a genipin crosslinked woven collagen mesh (CollaMesh) promotes de novo collagen deposition in amounts comparable to that attained by polypropylene slings at 5 months (9, 10), whereas EDC/NHS crosslinked woven collagen meshes did not result in robust healing.

Preparation of iridoid crosslinking agents, including genipin, loganin aglycone, oleuropein aglycone, or E-6-O-methoxycinnamoyl scandoside methyl ester aglycone, has been described (112). Genipin, loganin aglycone, oleuropein aglycone, or E-6-O-methoxycinnamoyl scandoside methyl ester aglycone can be prepared from Gardenia jasminoides Ellis, Lonicera japonica Thunb, Olea europaea Linn, and Hedyotis diffusa (Willd) Roxb, respectively (112).

The structural protein of the biologic material can be crosslinked by treatment with genipin dissolved in an aqueous ethanol solution, e.g., 90% ethanol, for example at 0.5 to 3.0% (w/v), 1.0% to 2.5% (w/v) or about 2.0% (w/v). For example, a mesh of the structural protein can be placed in such a genipin solution and incubated, for example, for 72 h at 37° C., to crosslink the structural protein.

Accordingly, in some embodiments, the crosslinked structural protein has been crosslinked with an iridoid crosslinking agent. In some embodiments, the crosslinked structural protein has been crosslinked with one or more of genipin, loganin aglycone, oleuropein aglycone, or E-6-O-methoxycinnamoyl scandoside methyl ester aglycone. In some embodiments, the crosslinked structural protein has been crosslinked with genipin.

As noted above, the biologic material can have a material structure corresponding to a scaffold, such as a mesh, or a viscous fluid, such as a gel, or a form intermediate between a scaffold and a viscous fluid, such as a spongy form, among other material structures.

The material structure of the biologic material is determined, predominantly or entirely, by the packing density and degree of crosslinking of the crosslinked structural protein. Relatively low packing densities and degrees of crosslinking are suitable for forming viscous fluids, such as gels, whereas relatively high packing densities and degrees of crosslinking are suitable for forming scaffolds, such as meshes.

Accordingly, in some embodiments the crosslinked structural-protein forms a crosslinked structural-protein scaffold, such as a crosslinked structural-protein mesh. Regarding crosslinked structural-protein meshes, in some embodiments the crosslinked structural-protein mesh comprises a crosslinked collagen mesh. The crosslinked collagen mesh can be, for example, a crosslinked electrochemically aligned collagen mesh. In some embodiments, the crosslinked collagen mesh comprises a crosslinked woven collagen mesh. The crosslinked woven collagen mesh can be, for example, a woven crosslinked electrochemically aligned collagen mesh.

Also accordingly, in some embodiments the crosslinked structural-protein forms a crosslinked structural-protein viscous fluid, such as a crosslinked structural-protein gel. Regarding crosslinked structural-protein gels, in some embodiments the crosslinked structural-protein gel comprises a crosslinked collagen gel.

As noted above, the biologic material also comprises macrophages seeded on the crosslinked structural protein. This means that macrophages are cultivated in vitro and then applied to the crosslinked structural protein prior to implantation of the biologic material into a human or animal. This allows for the macrophages to become attached to the crosslinked structural protein prior to implantation. This also allows the biologic material to introduce macrophages directly at the site of implantation of the biologic material in a human or animal. This also allows for the macrophages to be present at higher densities on the biologic material, throughout the process of collagen deposition, neovascularization, tissue integration, and healing, than for implants comprising the crosslinked structural protein not seeded with macrophages.

As discussed above, macrophages are central to the orchestration of repair and regeneration of healing tissues (32, 33), macrophages polarize mainly into M1 and M2 types which are proinflammatory and anti-inflammatory, respectively, and macrophages are accessible cells and their collection is facile for therapeutic purposes. Also as noted, it is believed that delivery of autologous-blood derived M2 macrophages and/or allogeneic-blood derived M2 macrophages via genipin crosslinked collagen meshes will result in faster regeneration; specifically, earlier deposition of collagen, earlier inception of neovascularization and earlier attainment of mechanical robustness than the collagen meshes which are implanted without M2 macrophages.

In some embodiments the macrophages comprise autologous-blood derived macrophages and/or allogeneic-blood derived macrophages. Autologous-blood derived macrophages refers to macrophages obtained from the same individual in which the biologic material is to be used. Allogeneic-blood derived macrophages refers to macrophages obtained from a donor other than the individual in which the biologic material is to be used. The donor can be, for example, either a matched related or a matched unrelated donor.

Also, in some embodiments the macrophages comprise M2 macrophages. In some examples, the macrophages can comprise M2 macrophages derived from monocytes. These can be obtained, for example, by treating the monocytes with a mixture of TGF-β, IL-4, IL-10, and/or IL-13 to obtain the M2 macrophages. Also in some examples, the macrophages can comprise M2 macrophages derived from M0 macrophages. These can be obtained, for example, by treating the M0 macrophages with genipin in a solution to obtain the M2 macrophages.

The macrophages can be seeded on the crosslinked structural protein of the biologic material at specific densities. The densities can be specified, for example, as number of macrophages per volume of the biologic material. The densities also can be specified, for example, as a percentage of confluence of the macrophages on the surface of the biologic material.

For a sample of the biologic material corresponding to a mesh sized to 25 mm×4 mm×0.25 mm to match the size of slings implanted in rats, 2.5×10⁵ macrophages per mesh is sufficient to cover the surface of the mesh with macrophages to about 25% confluence, and 1×10⁶ macrophages per mesh is sufficient to cover the surface of the mesh with macrophages to nearly 100% confluence. It is believed seeding macrophages at a density of 2.5×10⁵ macrophages per mesh so sized is sufficient for faster regeneration, following implantation, than without seeding. It also is believed that lower seeding densities, e.g., 1.25×10⁵ macrophages per mesh so sized, would be sufficient. It also is believed that seeding macrophages at a density of 1×10⁶ macrophages per mesh so sized, and thus to nearly 100% confluence, defines an upper boundary for seeding density. It also is believed that higher seeding densities, e.g., 2.5×10⁶ macrophages per mesh so sized, would be tolerated.

The seeding density of 1.25×10⁵ to 2.5×10⁶ macrophages per mesh sized to 25 mm×4 mm×0.25 mm corresponds to 5,000 to 100,000 macrophages/mm³ of mesh. The seeding density of 2.5×10⁵ to 1×10⁶ macrophages per mesh sized to 25 mm×4 mm×0.25 mm corresponds to 10,000 to 40,000 macrophages/mm³ of mesh.

Accordingly, in some embodiments the macrophages have been seeded on the crosslinked structural protein at a seeding density of 5,000 to 100,000 macrophages/mm³ of the biologic material. Also, in some embodiments the macrophages have been seeded on the crosslinked structural protein at a seeding density of 10,000 to 40,000 macrophages/mm³ of the biologic material. For example, in some embodiments the macrophages have been seeded on the crosslinked structural protein at a seeding density of 5,000 to 10,000 macrophages/mm³, 5,000 to 40,000 macrophages/mm³, 10,000 to 100,000 macrophages/mm³, or 40,000 to 100,000 macrophages/mm³ of the biologic material. Also, in some embodiments the macrophages have been seeded on the crosslinked structural protein at a seeding density of 10,000 to 20,000 macrophages/mm³, 15,000 to 25,000 macrophages/mm³, 20,000 to 30,000 macrophages/mm³, 25,000 to 35,000 macrophages/mm³, or 30,000 to 40,000 macrophages/mm³ of the biologic material. Also, in some embodiments the macrophages have been seeded on the crosslinked structural protein at a seeding density of 10,000 to 35,000 macrophages/mm³, 10,000 to 30,000 macrophages/mm³, 10,000 to 25,000 macrophages/mm³, 10,000 to 20,000 macrophages/mm³, or 10,000 to 15,000 macrophages/mm³ of the biologic material. Also, in some embodiments the macrophages have been seeded on the crosslinked structural protein at a seeding density of 15,000 to 40,000 macrophages/mm³, 20,000 to 40,000 macrophages/mm³, 25,000 to 40,000 macrophages/mm³, 30,000 to 40,000 macrophages/mm³, or 35,000 to 40,000 macrophages/mm³ of the biologic material.

Because macrophages are applied to the crosslinked structural protein of the biologic material prior to implantation, the biologic material has a distinctive composition and structure prior to implantation, in comparison to previous implants. For example, prior to implantation the biologic material has not yet been subjected to deposition of collagen, nor to neovascularization, nor to integration of tissue, nor to the presence of human or animal cells other than those seeded on the biologic material. Moreover, because the cells seeded on the biologic material are predominantly macrophages, prior to implantation, to the extent that human or animal cells are attached to the crosslinked structural protein of the biologic material, the human or animal cells are predominantly macrophages.

Accordingly, in some embodiments, particularly prior to implantation, the biologic material does not include any collagen deposited on the crosslinked structural protein. Also, in some embodiments, again prior to implantation, the biologic material does not include any neovascularization. Also, in some embodiments, again prior to implantation, the biologic material does not include any tissue integrated into the crosslinked structural protein.

Also, in some embodiments, again prior to implantation, to the extent that human or animal cells other than macrophages are present on the crosslinked structural protein of the biologic material, more macrophages are present than the other human or animal cells. For example, in some embodiments, to the extent that human or animal cells other than macrophages are present on the crosslinked structural protein, at least 51%, at least 60%, at least 70%, at least 80%, at least 90%, at least 95%, at least 99%, or 100% of the total number of human or animal cells present on the crosslinked structural protein of the biologic material are macrophages.

Also, in some embodiments, again prior to implantation, to the extent that human or animal cells other than macrophages are attached to the crosslinked structural protein of the biologic material, more macrophages are attached than the other human or animal cells. For example, to the extent that human or animal cells other than macrophages are attached to the crosslinked structural protein, at least 51%, at least 60%, at least 70%, at least 80%, at least 90%, at least 95%, at least 99%, or 100% of the total number of human or animal cells attached to the crosslinked structural protein of the biologic material are macrophages.

A method of use of the biologic material for an immunoregenerative treatment in a patient in need thereof also is provided. The biologic material is as described above. The method comprises steps of: (1) seeding the macrophages on the crosslinked structural protein, thereby obtaining the biologic material; and (2) implanting the biologic material into the patient.

In some embodiments, the macrophages comprise M2 macrophages derived from monocytes. In accordance with these embodiments, the method further comprises steps of: (0.1) treating the monocytes with Macrophage Colony Stimulating Factor to obtain M0 macrophages; and (0.2) treating the M0 macrophages with a mixture of TGF-β, IL-4, IL-10, and/or IL-13 to obtain the M2 macrophages.

In some embodiments, the macrophages comprise M2 macrophages derived from M0 macrophages. In accordance with these embodiments, the method further comprises a step (0.3) of treating the M0 macrophages with genipin in a solution to obtain the M2 macrophages.

In some embodiments, the method further comprises treating the macrophages with IL-4 during step (1).

In some embodiments, the immunoregenerative treatment comprises one or more of treatment of stress urinary incontinence, treatment of pelvic organ prolapse, hernia repair, or an orthopedic application.

In some embodiments, the crosslinked structural protein comprises one or more of a crosslinked collagen, a crosslinked gelatin, a crosslinked elastin, or a crosslinked keratin. In some embodiments, the crosslinked structural protein comprises a crosslinked collagen, e.g., a crosslinked electrochemically aligned collagen.

In some embodiments, the crosslinked structural protein has been crosslinked with an iridoid crosslinking agent. In some embodiments, the crosslinked structural protein has been crosslinked with one or more of genipin, loganin aglycone, oleuropein aglycone, or E-6-O-methoxycinnamoyl scandoside methyl ester aglycone. In some embodiments, the crosslinked structural protein has been crosslinked with genipin.

In some embodiments the crosslinked structural-protein forms a crosslinked structural-protein scaffold, such as a crosslinked structural-protein mesh. Regarding crosslinked structural-protein meshes, in some embodiments the crosslinked structural-protein mesh comprises a crosslinked collagen mesh, e.g., a crosslinked electrochemically aligned collagen mesh. In some embodiments, the crosslinked collagen mesh comprises a crosslinked woven collagen mesh, e.g., a woven crosslinked electrochemically aligned collagen mesh.

Also, in some embodiments the crosslinked structural-protein forms a crosslinked structural-protein gel. For example, in some embodiments the crosslinked structural-protein gel comprises a crosslinked collagen gel.

Another biologic material also is provided. The biologic material comprises (a) a crosslinked amine-functionalized biodegradable polymer; and (b) macrophages seeded on the crosslinked amine-functionalized biodegradable polymer. In some embodiments, the crosslinked amine-functionalized biodegradable polymer comprises one or more of a crosslinked amine-functionalized biodegradable polyester, a crosslinked amine-functionalized poly lactic-co-glycolic acid, a crosslinked amine-functionalized polycaprolactone, a crosslinked amine-functionalized biodegradable polysaccharide, or a crosslinked amine-functionalized chitosan.

The biologic material comprising a crosslinked amine-functionalized biodegradable polymer and macrophages seeded on the crosslinked amine-functionalized biodegradable polymer can be made and used similarly as for the biologic material comprising a crosslinked structural protein and macrophages seeded on the crosslinked structural protein as described above.

For example, in some embodiments the crosslinked amine-functionalized biodegradable polymer has been crosslinked with an iridoid crosslinking agent. In some of these embodiments, the crosslinked amine-functionalized biodegradable polymer has been crosslinked with one or more of genipin, loganin aglycone, oleuropein aglycone, or E-6-O-methoxycinnamoyl scandoside methyl ester aglycone. The amine functionalization of the biodegradable polymer allows the biodegradable polymer to be crosslinked by iridoid crosslinking agents such as genipin.

Also for example, the biologic material comprising the crosslinked amine-functionalized biodegradable polymer and macrophages seeded on the crosslinked amine-functionalized biodegradable polymer can have a material structure corresponding to a scaffold, such as a mesh, or a viscous fluid, such as a gel, or a form intermediate between a scaffold and a viscous fluid, such as a spongy form, among other material structures. In some embodiments, the crosslinked amine-functionalized biodegradable polymer forms a crosslinked amine-functionalized biodegradable polymer mesh. In some embodiments, the crosslinked amine-functionalized biodegradable polymer forms a crosslinked amine-functionalized biodegradable polymer gel.

Also for example, in some embodiments, the macrophages comprise autologous-blood derived macrophages and/or allogeneic-blood derived macrophages. In some embodiments, the macrophages comprise M2 macrophages. In some embodiments, the macrophages have been seeded on the crosslinked amine-functionalized biodegradable polymer at a seeding density of 5,000 to 100,000 macrophages/mm³ of the biologic material.

Also for example, in some embodiments, to the extent that human or animal cells other than macrophages are present on the crosslinked amine-functionalized biodegradable polymer of the biologic material, more macrophages are present than the other human or animal cells. In some embodiments, to the extent that human or animal cells other than macrophages are attached to the crosslinked amine-functionalized biodegradable polymer of the biologic material, more macrophages are attached than the other human or animal cells.

### EXAMPLES

### Example 1

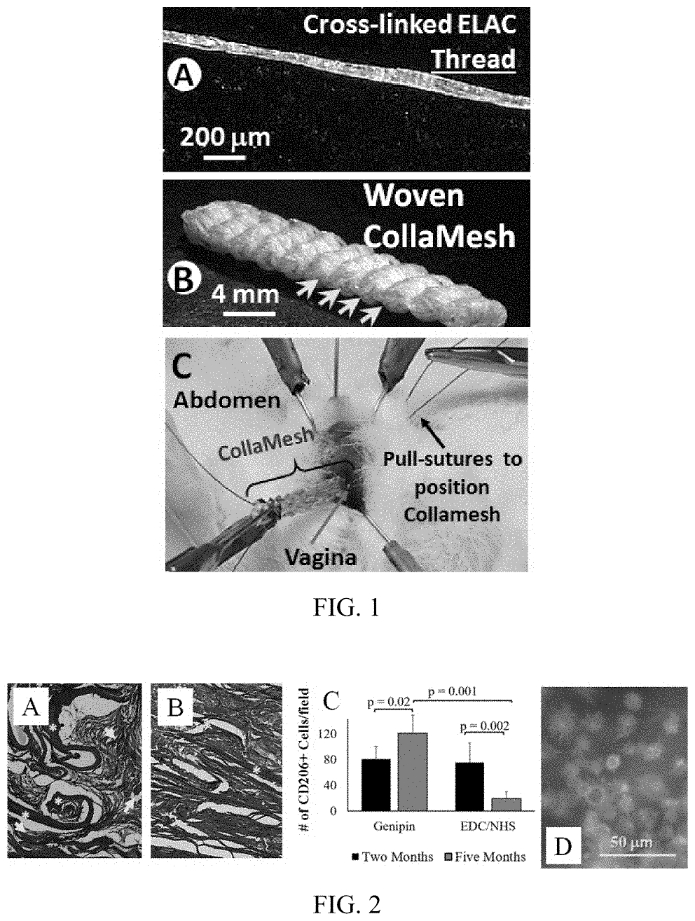

Innovation and Related Work: To our knowledge controlled delivery of blood derived M2 macrophages on a material platform that will act as an immunoregenerative tool to address SUI has not been achieved previously. We aim to attain this outcome on an innovative genipin crosslinked woven collagen biotextile (FIG. 1). Genipin is a plant-derived anti-inflammatory agent which we observed to promote M2-macrophages in its milieu in vivo (FIG. 2C) and also per the emerging literature (48-50). Macrophages readily adhere to genipin CollaMesh (FIG. 2D). Woven collagen mesh is a highly porous yet mechanically robust biotextile that has excellent tissue integration properties (10). From a translational perspective, a novel alternative to autografts may emerge from this high risk study. Genipin crosslinked woven collagen mesh will merge good qualities of polypropylene slings (superior tissue integration, long-term continence, de-novo collagen deposition) and xeno/autografts (low erosion/extrusion) to improve the patient care.

Electrochemical Compaction of Collagen as Mechanically Functional Scaffolds: Normally, reconstituted collagen has the consistency of a gel that cannot hold sutures or bear mechanical loads. We addressed this significant limitation by electrochemical compaction and alignment of collagen molecules (ELAC) (1, 51). Unlike electrospinning, collagen molecules are not denatured (52-54) during this process. A device to fabricate ELAC threads in continuous length which in turn can be woven as implantable slings that can hold sutures has been described (FIG. 1) (52). To our knowledge, CollaMesh is the only pure collagen form that is implantable as a functional sling (FIG. 1C and FIG. 4). The weaving pattern of CollaMesh results in two tiers of porosity which enable rapid integration of host tissue and vasculature. The first tier is the macroscale in-plane cylindrical porosity (>0.5 mm) which extends along the width of the sling (FIG. 1B). The second tier is mesoscale porosity (>150 microns) in the form of gaps between neighboring yarns. Pore network provides access to inflammatory system such that none of in vivo applications of CollaMesh resulted in infection (55, 56). These factors render CollaMesh suitable for engineering a urethral sling that is mechanically functional.

Genipin crosslinking of CollaMesh enhances de novo collagen deposition in vivo: Tissue integration, mechanical properties and M2-macrophage counts on subcutaneously implanted CollaMesh (crosslinked with EDC/NHS vs. Genipin) were compared to XenMatrix (Bard, porcine dermis xenograft) and Prolene (Ethicon, polypropylene mesh). All articles (N=5/material/time-point) were implanted abdominally in rats. Samples were harvested at 2 weeks, 2 months and 5 months and tested in tension immediately after dissection. Modulus (material level stiffness) was calculated from stress-strain curves and compared to the modulus of vagina and abdominal fascia dissected from rats (9, 10). Separate set of samples were stained in hematoxylin eosin for quantification of tissue biocompatibility (in accordance with ISO 10993-6), and, Masson trichrome for de novo collagen formation. Anti-CD206 (Santa Cruz) antibody was used to immunostain 2 month and 5 month timepoint sections for M2-macrophages.

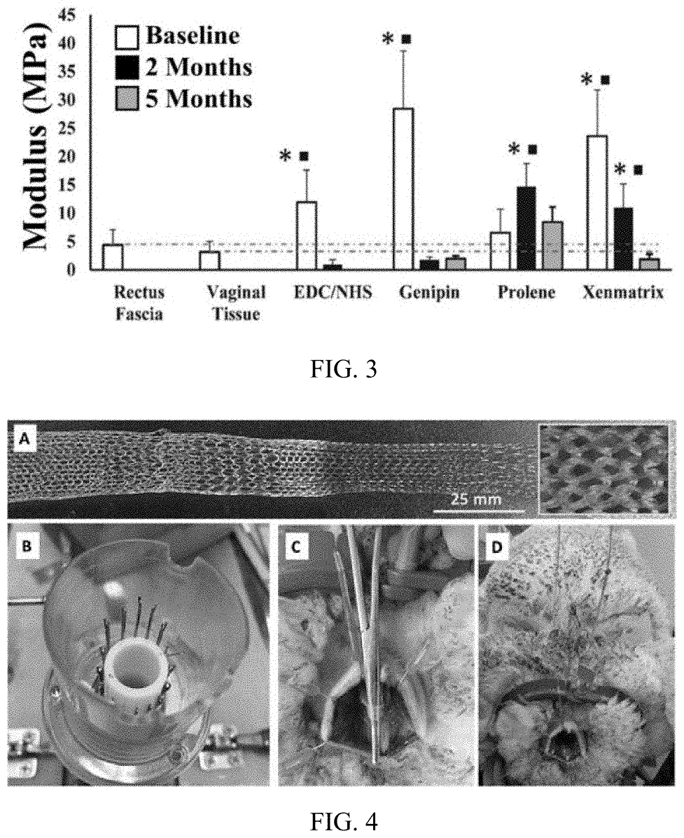

Results: Tissue integration in xenograft dermis was absent (data not shown) while the open network of woven collagen (FIG. 2) and Prolene meshes allowed for cellular and tissue integration at all time-points. Collagen deposition scores (per ISO10993-6) were comparable between genipin and Prolene meshes (3.5±0.5 and 3.75±0.5, respectively, p>0.05, moderate to extensive presence) at 5-months whereas collagen deposition in EDC/NHS meshes were limited (2±1, mild presence). Genipin meshes attracted a greater number of M2 macrophages than ECC meshes at 2 and 5 months (FIG. 2C). The modulus (FIG. 3) of genipin crosslinked collagen, Prolene and Xenmatrix meshes matched that of native rectus fascia and vaginal tissues at five months, while EDC/NHS crosslinked meshes were degraded so extensively that 4 out of 5 samples were not recoverable at five months for tensile tests.

Discussion: These results indicate that the woven nature of genipin formulation favors tissue integration and collagen deposition that is similar to the standard of care polypropylene. It appears that favorable response to CollaMesh is driven by the crosslinker genipin because EDC/NHS crosslinked collagen mesh did not result in a mechanically viable repair. Another key difference between genipin and EDC/NHS crosslinking was such that genipin crosslinking attracted anti-inflammatory M2 phenotype macrophages. Genipin, a natural crosslinker derived from jasmine plant, is known to have anti-inflammatory effects in Asian medicine (57). Scientific studies have also shown genipin to have anti-inflammatory effect on macrophages in vitro and to reduce inflammation in vivo (48-50). Such literature aligns with the observations reported here.

Conclusion: Genipin crosslinked CollaMesh holds promise as a new sling biomaterial based on favorable tissue integration, induction of fibrous tissue formation and in vivo mechanical stiffness that is matching those of native tissue at 5 months. On the other hand, there is a steep decline in the mechanical properties of collagen meshes between 0-2 months indicating that the healing timeline, specifically collagen deposition can be expedited to occur at earlier time-points than 5 months. Delivery of peripheral blood derived M2-macrophages at the baseline as proposed in this study will expedite and improve the healing.

### Example 2

Approach: Our overall approach (Table 1) will be such that genipin crosslinking level and macrophage seeding density that will provide improved regeneration will be identified in Aim 1 via in vitro assays and subcutaneous implantation of NU-macrophage loaded CollaMesh formulations. In case genipin has limitations in maintaining M2-polarization, we will also assess IL-4 delivery from CollaMesh to enhance polarization status of cells. Subcutaneous implantation of CollaMesh in Aim 1 will also determine effective cell-seeding density. The formulation identified in Aim 1 will be used as a functional urethral sling in Aim 2 to compare the proposed approach's efficacy to that of polypropylene repair as a function of time.

Aim 1: Enhance immunoregenerative capacity of M2 macrophages seeded on CollaMesh.

These studies will identify a mesh formulation and cell seeding density to be used in Aim 2 studies. Monocytes will be collected from blood, induced to M2 macrophages, seeded on collagen meshes that are crosslinked with low and high doses of genipin. Genipin CollaMesh will be heparinized to deliver IL-4 in an additional group, a potent inducer of M2-polarization, to determine whether IL-4 supplementation further enhances the polarization state and production of pro-regenerative factors. Prolonged and sustained delivery of cytokines from heparinized collagen has been described (58) and will be adopted for IL-4. In vitro studies will identify genipin dose will be anti-inflammatory, profibrogenic and proangiogenic and whether IL-4 delivery be essential in ensuing stages of Aim 1 during which effects of cell seeding density on in vivo collagen production and vascularization will be determined by subcutaneous implantation of autologous M2 macrophages in rats.

TABLE 1
Timeline and activities.

| | | Year 1 | | | | | | Year 2 | | | | | |
|---|---|---|---|---|---|---|---|---|---|---|---|---|---|
| Step | Activities | (Months) | | | | | | (Months) | | | | | |
| 1. Identify a | Fabrication of CollaMesh | X | | | | | | | | | | | |
| genipin | Assess effects of genipin | | X | | | | | | | | | | |
| formulation and | crosslinking dose and IL-4 delivery | | | | | | | | | | | | |
| macrophage | on maintaining M2 macrophage | | | | | | | | | | | | |
| seeding density | phenotype in vitro | | | | | | | | | | | | |
| that will | Assess effects of M2-macrophage | | | X | X | X | | | | | | | |
| improve | seeding density and IL-4 delivery in | | | | | | | | | | | | |
| collagen | vivo (rat subcutaneous) | | | | | | | | | | | | |
| deposition and | Mechanical tests | | | | X | X | | | | | | | |
| scaffold | Quantitative histo- and | | | | X | X | X | | | | | | |
| mechanics in | immunohisto-analysis of collagen | | | | | | | | | | | | |
| vivo. | deposition and vascularization | | | | | | | | | | | | |
| 2. Compare | CollaMesh fabrication | | | | | X | X | | | | | | |
| functional | Macrophage seeding + surgery + | | | | | | | X | X | | | | |
| outcome of | animal care (2 months) | | | | | | | | | | | | |
| CollaMesh to | Macrophage seeding + surgery + | | | | | | | X | X | X | | | |
| autograft and | animal care (5 months) | | | | | | | | | | | | |
| polypropylene. | Leak point pressure and tissue | | | | | | | | X | | X | | |
| | harvest | | | | | | | | | | | | |
| | Quantitative histo- and | | | | | | | | X | X | X | X | X |
| | immunohisto-analysis | | | | | | | | | | | | |
| | Data reduction, analysis, manuscript | | | | | X | X | | | | | | |
| | preparation | | | | | | | | | | | | |

Sub Aim 1.1 Assess Effects of Genipin Dose and IL4 Delivery on M2 Polarization In Vitro.

Collection of blood from donor rats and monocyte isolation for in vitro tests: Isolation of macrophages from tissues and blood has been described (41, 42). For in vitro studies, blood will be collected from the left ventricles of donor rats. 100 mL of blood will be collected from five rats which will provide 25×monocytes in total (43). Collected blood will be diluted with PBS and EDTA coagulant. Monocytes will be collected by density gradient centrifugation on Ficoll-Hypaque followed by selection of CD14+ monocytes by a detection kit using a magnetic cell sorter (Miltenyi Biotec).

Polarization of monocytes to M2 and M1 types (44): Monocytes (2.5×10⁵/mL) will be cultured overnight in RPMI 1640 medium supplemented with glutamine, mercaptoethanol, 10% FBS, penicillin, and streptomycin and induced to M2 macrophage lineage by supplementing the medium with a mixture of TGF-β, IL-4, IL-10 and IL-13 (each at 20 ng/mL) for 24 hours. This formulation induces robust polarization to M2 lineage (44). A separate group of monocytes will be polarized to inflammatory M1 macrophages by supplementing the medium with LPS and IFNγ for 24 h as a control group.

Confirmation of macrophage types: Following 24 hours, cells will be stained for the following antibodies: CD14 (M0), CD80 (M1), CD86 (M1), CD163 (M2) and CD206 (M2) along with markers of dead cells (Life Technologies) and subjected to flow cytometry (Beckman Coulter, CA, USA). Median fluorescence intensity (MFI) of markers will be measured and isotype controls will be used to adjust for background signals.

Fabrication of CollaMesh: ELAC threads are produced in continuous length, using a rotating electrode electrochemical thread production device (REEAD) as previously described (60). Genipin crosslinking will be performed using ethanol:water (80:20 v/v) for 24 hours as previously described (51, 61). Scaffolds will be sized to 25×4×0.25 mm to match the size of the slings implanted in rats as shown in FIG. 1B-C.

Sustainment of macrophage polarization and production of profibrogenic and proangiogenic factors on CollaMesh: M2-macrophages polarized as described above will be seeded on the following material formulations to determine whether M2 polarization is sustained. The following treatment groups will be included: 1) Uncrosslinked CollaMesh (i.e. 0% genipin) with M2 macrophages, 2) CollaMesh crosslinked in 0.625% genipin (low genipin dose) with M2 macrophages, 3) CollaMesh crosslinked in 2% genipin (high genipin dose) with M2 macrophages. It may also be possible that inclusion of M1 macrophages along with M2 macrophages may increase fibrogenic and angiogenic factors. Therefore, we will include additional groups where 0.625% genipin crosslinked meshes will be seeded with M1 macrophages (Group 4) or 50/50 mixture of M1 and M2 macrophages (Group 5). Heparinized CollaMesh crosslinked with 0.625% genipin will be loaded with IL-4 to determine whether this cytokine enhances the response (Group 6). Sustainment of anti-inflammatory M2 phenotype will be assessed by spiking the all treatment groups by LPS and IFNγ to induce inflammatory response. Cells seeded on culture plates without LPS and IFNγ will be used to normalize the measurements to obtain fold-increase in response with respect to controls which are not stimulated.

Inflammatory response: The inflammatory response will be quantified by the production of inflammatory (IL-6, TNF-α) and antiinflammatory (IL-10, TGF-β) markers in the culture medium by ELISA at 24 h, 3 days and 7 days. The inflammatory response also will be assessed by endocytosis using a kit (pHrodo Red Dextran, 10,000 MW, ThermoFisher). Flow cytometry will be conducted as described earlier to count the number of M1 and M2 macrophages at each time point. We will be measuring the production of pro-fibrogenic and proangiogenic factors by cells in each treatment group, specifically VEGF, PDGF-BB, FGF-2 and IGF-1 by ELISA.

Expected results and alternative approaches: We expect the productions of VEGF, PDGF, FGF-2, IGF-1, IL-10 and TGF-β for genipin crosslinked Groups 2 and 3 to be greater than the other groups whereas IL-6 and TNF-α productions will be lower for genipin Groups 2 and 3. These changes may be more pronounced for the group crosslinked at the highest genipin concentration (Group 3). The fraction of M2 macrophages as determined by flow cytometry will be the greatest for 2% genipin crosslinked group at Day 7 time-point. It may be possible that the group with the highest anti-inflammation may not have the highest production of profibrogenic and proangiogenic factors. It is also possible that co-delivery of M1 and M2 to have a more potent effect than the delivery of M2 only (via comparison of groups 2, 4 and 5). If such will be the case, we may choose the group that provided the highest level of profibrogenic and proangiogenic factors. If IL-4 delivery per group 6 increases regenerative cytokine production then heparinized CollaMesh may be chosen in the ensuing stages. Power analysis is described below.

Sub Aim 1.2 Determine Effective M2-Macrophage Seeding Densities In Vivo.

M2 macrophage delivery has not been executed to date for tissue engineering purposes. These experiments will be useful for determining seeding densities of M2-macrophages that will have a regenerative effect in vivo. These experiments will also allow us to execute and refine autologous macrophage delivery approach prior to the main studies proposed in Aim 2, Blood will be collected autologously from lateral tail veins of each rat one week prior to implantation of scaffolds. This method allows for collection of blood up to 1 mL and for up to 4 times a week, providing up to 500,000 monocytes. Therefore, two collections will provide the 1×10⁶ cells for the highest seeding density group. An amount of 1×10⁶ cells/scaffold is sufficient to cover the surface of an implant to near confluence, defining the upper bound for seeding density. There will be five treatment groups: Group 1: Genipin CollaMesh only, Group 2: Heparinized Genipin CollaMesh with IL-4, Group 3: Genipin CollaMesh seeded with 2.5×10⁵ cells/scaffold, Group 4: Heparinized Genipin CollaMesh with IL-4 seeded with 2.5×10⁵ cells/scaffold, Group 5: CollaMesh seeded with 1×10⁶ cells/scaffold. Each rat will be implanted with 4 discretely positioned implants of the same type and there will be 5 rats per group per time point, resulting in 20 specimens/group/timepoint. Animals will be euthanized at 2 and 5 months, latter of which is sufficiently long for neovascularization and collagen deposition to take place per our preliminary data. By two months we have seen substantial decline in implant stiffness in this model (FIG. 3) and inclusion of this early time point is going to provide us with evidence whether or not cell seeding curbs such decline. Twelve specimens will be tested mechanically and 8 specimens will be histologically processed for quantitative histological scoring and immunostaining as described in the preliminary data section.

Expected results: Collagen deposition and neovascularization will start earlier and will increase with increasing number of macrophages delivered. This outcome will be associated with a less steep decline in the mechanical properties of scaffolds. It is possible that highest cell seeding density group may be associated with extra stiffness (defined by 2-fold or greater stiffness than that of vagina, 10 MPa+). In that case, we may opt for Group 2 for Aim 2 studies, We may also opt for IL-4 delivery based on the outcome from Groups 2 and 4.

Aim 2: Evaluate the Effects of M2 Macrophage Delivery on In Vivo Functional Outcome on Stress Urinary Incontinence Treatment.

Why rat SUI Model? We chose the SUI model in which rats will be rendered chronically incontinent by pudendal nerve transection as previously described (62-64). PNT in primates have shown incontinence for up to a year of follow up (69); however, primates would be prohibitively costly and premature for this study. Other than primates, the proposed rat model has the longest incontinence duration of up to 3 months (65-67). We expect incontinence in rat PNT model to be durable at 5 months because we are completely resecting the nerve at a size that prohibits re connection and repair.

Treatment groups: 1) incontinent-untreated (control), 2) CollaMesh formulation (cell density and IL-4 delivery determined per Aim 1), 3) polypropylene mesh (PROLENE, Ethicon) as the standard material in SUI treatment. The mesh has keyhole shaped pores (˜0.5×1 mm to 7×20 units of pores along width and length, respectively). There will be 20 rats/group.

LPP, vaginal/urethral wall thicknesses (measure of tissue atrophy), healing stiffness and quantitative histological assessment of de novo tissue formation will be performed. There will be two time points at 2 months and 5 months to evaluate early and mature phases of remodeling. Earlier studies assessed sling materials subcutaneously in the abdomen up to 1 year and in such setting the material response converges a steady state by 6 months (70). Functional sling models to date are limited to within 3 months; thus, proposed 5 month duration will be one of the longest among SUI models, sufficient to assess mature phase of remodeling.

Induction of SUE and sling implantation: Female Sprague-Dawley rats (250-300 grams) will be used for the SUI model as previously described by our group (62-64) by bilateral pudendal nerve transection. Leak Point Pressure Testing: LPP testing will be performed prior to euthanasia as described (62-64). LPP will be tested 10 times in each rat.

Mechanical properties: Slings will be dissected and tested mechanically immediately after euthanasia as described above. Modulus, failure strength and failure stresses will be calculated. Ten specimens will be spared for measuring biomechanical properties.

Quantitative histology for atrophy: Vaginal and urethral thickness measurements will assess tissue atrophy. Prior to histological embedding, urethral catheters will be placed in urethra and vagina to delineate and support these orifices against collapse during histological processing. The wall thicknesses will be measured at 10 equidistant cross sections and in each section, thickness will be measured at 45 degree increments around the circumference to ensure full coverage. The degree of inflammation, vascularity and the type/amount of de novo connective tissue within the mesh will be measured from hematoxylin-eosin or Masson's trichrome stained sections. Collagen organization will be scored after Badylak et al. (71). A moderate or high collagen content that is cellular and vascular to the extent of rat fascia will be considered a success. Immunostaining for macrophage phenotypes and myofibroblasts will be conducted as described (10).

Proof-of-concept experiments in ewes: The rat model is ideal to identify a formulation and approach cost effectively within this study. However, long term success of the CollaMesh concept depends on demonstrating feasibility in large animal models. We recently developed a labscale knitting machine (FIG. 4B) that can knit collagen yarns as slings (FIG. 4A). Polypropylene and macrophage seeded CollaMesh slings comparably sized to those used in humans (30 cm×15 mm×0.2 mm) will be implanted in ewes (FIG. 4) (N=3/group). At 5 months, slings will be recovered, strips will be sampled for immunohistological assessment and mechanical tests as described above for rats.

Expected results and alternative approaches: We have successfully implanted CollaMesh slings and demonstrated patency stably under the urethra for up to 6 months (data not shown) (10); thus, we do not foresee problems such as sling migration. We expect the LPP attained by cell-seeded CollaMesh group to match that attained by polypropylene at 5 months. It is possible that cell-seeded CollaMesh formulation to have superior LPP than that of polypropylene at 2 months due to earlier inception of matrix deposition under the effect of M2 macrophages. A reduction in vaginal thickness is expected for the Prolene group whereas the reduction will be absent or less pronounced for the rest of the groups.

Data analysis: The experimental design in Aim 2 is a two-way design (different sling types, two time points). If data are normal per Anderson-Darling normality test, two-way ANOVA, (Tukey's post hoc) with or without data transformation will be performed. As needed, a nonparametric procedure, such as Friedman's test (Man Whitney post hoc with Bonferroni corrections), may also be performed. The sample size (12/group for mechanical or LPP tests) allows for detecting differences of more than 17% of the mean between different groups at 80% power assuming that standard deviation will be 20% of the mean. This magnitude of standard deviation is accordance with our prior experience for mechanical data and LPP data. Furthermore, rat SUI data as previously described (62-64) indicate that percent improvement in LPP with placement of sling with respected to incontinent controls to be more than 20%, therefore, the targeted 17% will be sufficient to resolve improvements with respect to incontinent treatment group. A sample size of N=8 for quantitative histological analysis will resolve 23% variation, again assuming 20% standard deviation and 80% power.

Vertebral Animal Studies:

Point 1: Species, sex, number and age of animals: We will use 6th to 8th week old female Sprague-Dawley rats in the proposed main studies. During the main study, specimens will be retrieved at 2 months and 5 months. Total number of rats will be 55 animals for Aim 1 (50 rats for subcutaneous implantation: 5 rats/group×5 groups×2 time points and 5 blood donors) and 160 animals for Aim 2 (20 rats/group×4 groups×2 time-points), total=215 animals. The statistical power provided by this sample size is discussed in the Section 3.3 of the grant proposal.

For the pilot proof-of-concept experiment, we will use 5th to 6th months old female Ewes. Total number of animals will be 6 animals (3/treatment group×2 groups×1 time point).

Point 2: Animal use justification for rats in the main studies: Currently there are no known non animal models that will allow us to test our hypothesis that the M2 enhanced CollaMesh attained by collagen electrocompaction will provide comparable functional outcomes to the current slings in addition to less morbidity and neo tissue formation. The scientific/technical reasons for the choice of this animal model is that it is an established model for SUI in which we have published experience. Furthermore, the rats are sufficiently sized and priced to test our hypothesis in the context of a proof of concept study. We will use rat SUI model by PNT method because rat is suitably sized to implant a mesh material that is still in the macroscale. PNT-SUI rat model is also well-established in the prior SUI tissue engineering/biomaterial literature (62, 63, 72). Overall, PNT model will help test our hypothesis while accommodating an acceptable statistical power.

Animal use justification for the pilot proof-of-concept experiment: Small animal models (e.g. rodents and rabbits) could be efficiently tested for undersized sling materials. In pigs, there are difficulties with the location of the urethral meatus. Pig presents a vaginal antechamber which makes the procedure not viable. The female dog had also been used previously, but it was abandoned due to its small vagina. Sheep model was carried out in literature as a model for learning of sling techniques because of its anatomical similarity in the aspects of human vagina. Reports of genital prolapse in elder sheep were described favoring the similarity with human anatomy and physiological support. We could not find a superior model for functional, biological and mechanical assessment of incontinence slings.

Point 3: We have a standing IUCAC approvals for a similar procedure in rat and sheep. A separate protocol/amendment will be secured for this project at CWRU. We also have past experience in working with rats and sheep. The Animal Research Center (ARC) of CWRU is AAALAC accredited for surgery, housing and care of animals. Veterinary doctors and technicians are on staff 24/7/365. They also provide training to the research staff. Animals will be ordered via an accredited vendor and will be acclimated for a week before being included in the study. Their access to food and water will be ad libitum. Animals will be attended during postsurgical recovery and frequently monitored to confirm that they resume feeding.

Point 4: General anesthesia procedures for rats: All surgeries and tests, including PNT, mesh insertion and LPP test will take place under anesthesia. For PNT and mesh implantation, rats will be anaesthetized by ketamine (100 mg/kg body weight) and xylazine (15 mg/kg body weight). While in suprapubic tube insertion and LPP test, a single intraperitoneal injection of urethane (1.2 gm/kg) will be used.

Subcutaneous implantation in rats: While under anesthesia, a sharp longitudinal midline incision about 5 cm will be done, then subcutaneous dissection bilaterally to create a space above the dorsal region. Implantation of each mesh material into the 4 different positions (upper right, upper left, lower right, lower left). Fixation of the implanted meshes with non-absorbable monofilament sutures from the sides. The skin then will be closed with skin staples which is removed after 1 to 14 days.

Surgical procedures for induction of incontinence and sling placement in rats: The vaginal sling implantation procedure in the rat are based on Hijaz model (62). All animals will be subjected to a procedure of SUI induction by pudendal nerve transection; isolation through bilateral dorsal longitudinal incision in the ischiorectal fossa, then removal of segment of nerve mm long) site to prevent neuro-regeneration. After closure of incision, urethral catheterization will be performed with PE-50 catheter. For the treatment groups, the anterior vaginal wall will be exposed and the mid urethral region accessed via an incision after hydro distention with 0.3 ml saline. The slings will be positioned using special grasping curved needles introduced abdominally through the pelvis. The techniques of sling placement are similar to the methods of clinical transvaginal sling placement. All procedures will be performed under complete aseptic conditions according to a protocol approved by the Institutional Animal Care Committee. After two or five months, all animals will undergo placement of a suprapubic tube under anesthesia and LPP testing.

Leak Point Pressure (LPP) assessment in rats: Cystometry and LPP testing will be performed under anesthesia as described by Damaser (73). Rats will be placed supine at the level of zero pressure with empty bladder. Then we fill bladder with saline at room temperature (5 ml per hour) through a suprapubic catheter, while bladder pressure is recorded. The suprapubic catheter is connected to a syringe pump (Kent Scientific, Litchfield, Conn.) and a pressure transducer (Grass Instruments Division, Astro-Med, West Warwick, R.I.). Pressure and force transducer signals are amplified (Grass Instruments Division) and digitized for computer data collection using computer software (BioBench, version 1.2, National Instruments Corp., Austin, Tex.). LPP will be tested at least 10 times in each rat.

Surgical procedures for sling placement in ewes: Ewes are given Isoflurane Midazolam at veterinary discretion for anesthesia. Lower abdomen and vagina are sterilized. This procedure will be similar to human TVT surgery. Using sterile instruments, a 1 cm surgical incision of the posterior vaginal wall (episiotomy) may be performed to enlarge the vaginal introits. Sterile Foley 12F urethral catheter will be inserted for bladder drainage and to identify the bladder neck. Sterile vaginal specula will be used to promote adequate retraction of the wall. Lidocaine will be injected prior to any incision, incisions will be made as the following: 1. One 3 cm median longitudinal anterior vaginal incision, 1 cm below the urethral meatus. 2. Two suprapubic 1 cm-size skin incisions; 5 cm apart, 1 cm above the pubis of the animal. The anterior vaginal wall overlying the urethra will be incised to expose the mid-urethra. Special prong needle will travel intra-abdominal through the suprapubic 1 cm-skin incisions on each side, in a downward motion with surgeon's forefinger acting as a guide (from vagina) during insertion from the suprapubic region to the vagina. One prong needle will be passed through each incision, into the rectus muscle and through the retropubic space, hugging the posterior aspect of the symphysis pubis, then perforating the endopelvic fascia and exiting through the already made vaginal incision.

A 20.0×1.0 cm sling attached to a 4-0 prolene suture will be prepared to make a hammock shaped sling. The prolene thread will be connected to the special needles' end on both sides and pulled to the suprapubic skin incision (from inside) via the needle passer placing the sling under the urethra. We will adjust the suburethral part to be tension-free. Closure of vaginal incisions will be done using 0 absorbable monofilament sutures. Abdominal wall will be closed with an appropriately sized absorbable suture. Skin will be closed with a subcuticular suture layer (2-0 Vicryl or equivalent) and surgical staples. Animals may wear abdominal wraps to avoid post-operative abdominal burst. Surgical staples will be removed within 14 days of surgery.

Point 5: Euthanasia, tissue harvest and analyses: Rats will be euthanized using exsanguination under anesthesia or cervical dislocation in line with the AVMA guidelines. Ewes will be euthanized using Pentobarbital Euthanasia Solution in line with the AVMA guidelines. Immediately after Euthanasia, we will collect the urethra, vagina and sling with surrounding tissue in case of suburethral implantation. We will collect the mesh and surrounding tissue in case of subcutaneous implantation. Tissue and biomaterials will be harvested for histopathological or mechanical examinations as detailed above.

Blood derived M2 macrophage delivery may change the state of the art in the treatment of patients who are contraindicated to polypropylene slings. Other than SUI, the proposed technology may impact a wide ranging clinical problems such as hernia repair, pelvic prolapse, cardiac repair patches and other soft tissue reinforcement applications.

### Example 3

Immunoregenerative Treatment of Stress Urinary Incontinence by Bone-Marrow Derived Macrophages Delivery Via Collagen

Genipin crosslinking of Collagen Mesh enhances de novo collagen deposition in vivo as

described previously (74). It has been reported that genipin meshes attract a more significant number of M2 macrophages at two and five months (74). This work highlighted the effect of genipin crosslinking treatments on the regenerative response and illustrates the importance of understanding the complex interactions of macrophages with collagen scaffolds.

Major Activities:

Materials and Methods

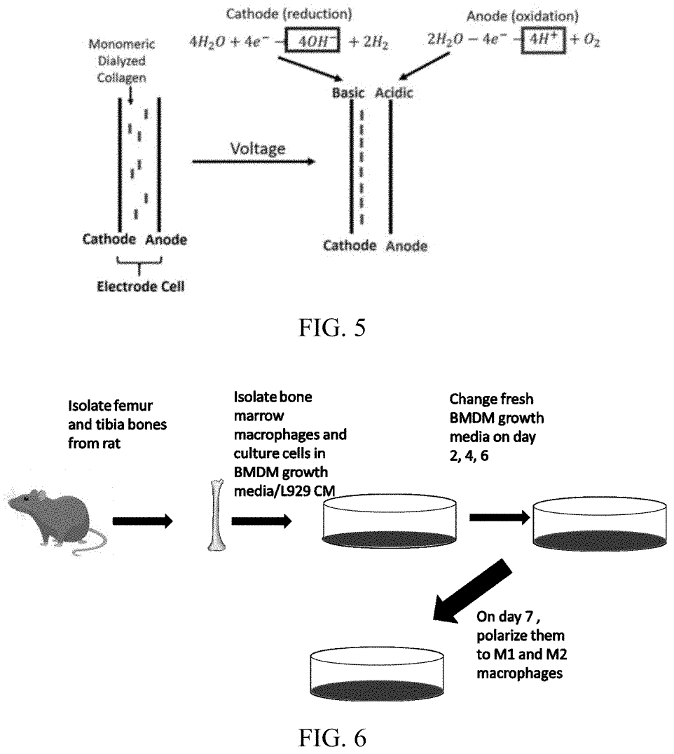

Fabrication of Collagen Scaffolds: Electrochemically aligned collagen (ELAC) threads, made as previously described (1), were crosslinked and woven into meshes. Acid solubilized collagen molecules (3 mg/mL) are dialyzed against deionized water for 24 hours. Following dialysis, the collagen solution was applied between two stainless steel electrode wires (1.8 mm separation). Electrical currents generate a pH gradient in the electrochemical cell. Collagen molecules acquire positive charge near the cathode and negative charge near the anode. Electro repulsion by electrodes compact the collagen molecules at the isoelectric point of the electrochemical cell where the net charges of the collagen molecules are zero (FIG. 5). A rotatory wheel device based on this principle was used to fabricate the aligned collagen threads in continuous length (1). The diameters of the threads were 0.11±0.03 mm. The collagen threads were then woven around pins with 1 mm diameter. Acidic collagen solution was applied on the scaffold as a glue to stabilize woven thread together.

The woven scaffold was crosslinked in genipin solution (0.625% w/v in 90% ethanol solution in distilled water) for 72 hours. The scaffolds were washed with deionized water, dehydrated and stored at 4° C. until use (FIG. 8A).

All meshes were sterilized in a 0.1% peracetic acid (PAA) solution. This sterilization method was chosen as it has been approved by the FDA for sterilization of medical devices and has been shown to incite minimal damage on collagenous tissues after treatment, although it does remove some of the color from genipin crosslinked threads (FIG. 8B). All meshes were rinsed several times with sterile 1×PBS and stored overnight at 4° C. before applications.

Animals: A total of fifteen eight-week-old female Sprague-Dawley (SD) rats (220-250 g) were used in this study. All surgical procedures and post-operative animal care were in accordance with the Guide for the Care and Use of Laboratory Animals (National Research Council, 1996) and the Guidelines and Policies for Rodent Survival Surgery provided by the Animal Care and Use Committees of Case Western Reserve University.

A method for isolation and growth of rat bone derived macrophages is provided in diagram form in FIG. 6.

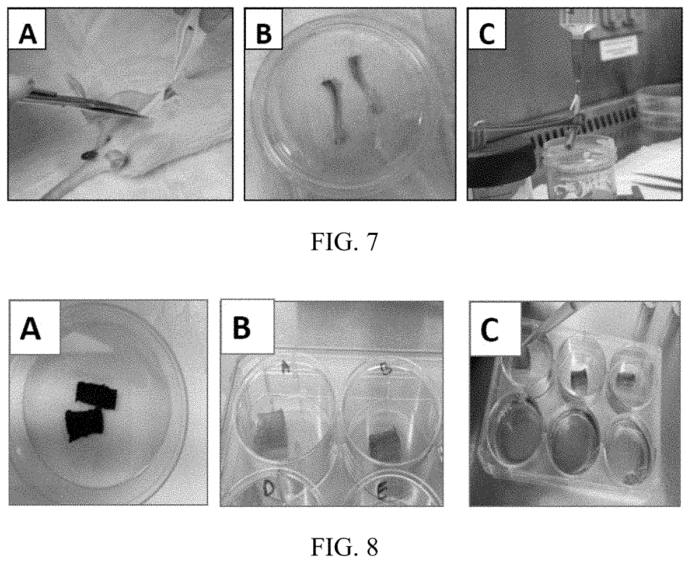

Isolation of Bone-Marrow Derived Macrophages: An equal number of control and treated rats were selected for isolation of bone-marrow derived macrophages (also termed BMDM). Falcon tubes were prepared with ˜20 mL DPBS (Life Technologies, Invitrogen™) (per XL) (1 tube per rat). Rats were euthanized with carbon dioxide in the animal facility. A midline incision was applied to expose intraabdominal organ in rats (FIG. 7A). Superficial fascia lateral and medial to quad muscles and full skin inferiorly to reveal underlying muscle was removed. A scissor was placed at the base of femur/at joint insertion into the pelvis and cut to isolate femur and repeated on another leg. The bones were immersed in 75% ethanol for 5 min, then soaked them in DPBS for 5 min and left them in DPBS+ Penicillin/Streptomycin (Life Technologies, Invitrogen™) until the next step. Bones with some muscles attached were dropped into PBS. Working quickly, samples were brought to tissue culture hood for no longer than 1 hour. In the sterile field, the remainder of muscle off of bone was cleaned with Kim wipes. Both ends of bone carefully were cut to reveal marrow space (large enough to fit 23 G gauge needle). A 23 gauge needle was placed on 10 mL syringe filled with ice-cold PBS and poured ˜20 mL complete media (10% FBS in DMEM) into a centrifuge tube. Cell clump contains RBCs, granulocytes, etc. For this reason, media was carefully poured off and resuspended in 30 mL L929 enriched media (1:4 of L929 media:10% FBS in DMEM). There may remain clumps of marrow. Bone marrow cells were cultured for 5-6 days. During this time, RBCs and non-macrophage cells will die, leaving the macrophage population for study. To lift cells, ice cold Ca2+ free PBS was used. The cells were counted using a hematocytometer (should have ˜25 million/15 cm dish) and plated ˜3 million cells per 1 well of 6 well plates (10 mm2 dishes). Non-enriched media (10% FBS in DMEM) was used. At this point, cells were treated and used for the study (cells viable for ˜3 days). The medium was changed every three days. While in culture, some of the cells become attached, while many of them still grow in suspension, so those cells were spun-down and recultured in new dishes.

Preparation of L929 conditioned medium: L929 cells (ATCC no. CCL-1) were cultured in DMEM/F12-10 (Invitrogen) until confluent. L929 cells were cultured ˜3 days and media collected and stored in −20° C. This is mixed in 1:4 concentration with 10% FBS) (Atlanta Biologicals) in DMEM to provide macrophage growth factors including M-CSF. L-929 conditioned medium was then harvested at confluence. Cells were removed by centrifugation, and supernatants were stored at −80° C. until use. Conditioned media was not freeze-thawed after this point, so single use aliquots (40 mL is enough for 160 mL total media to use on BMDM cells, which is the amount for 4 rats) were made.

Culture of Bone Marrow Derived Macrophages: After about 7 days, almost all BMDM cells became attached, and then the macrophages were used for further culture and tests. The above cells were defined as M0 macrophages. After 24-h incubation with 100 ng/ml LPS (Sigma, St. Louis, Mo.) and 25 ng/ml IFN-γ (R&D systems, Minneapolis, Minn.), the M0 cells were polarized into M1 cells. For M2 polarization, the M0 cells were incubated with 10 ng/ml IL-4, IL-10 (R&D systems) and TGF-transforming growth factor (TGF)-beta (R&D systems) for 24 h.

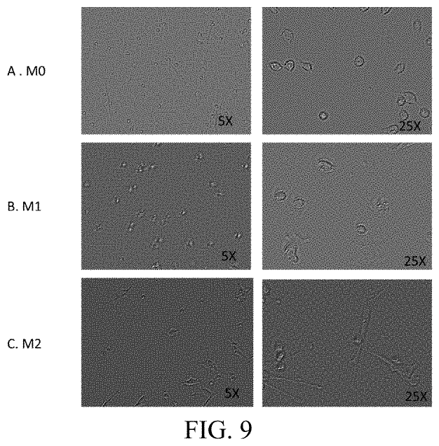

Morphotypes of polarized bone-marrow derived macrophages are shown in FIG. 9.

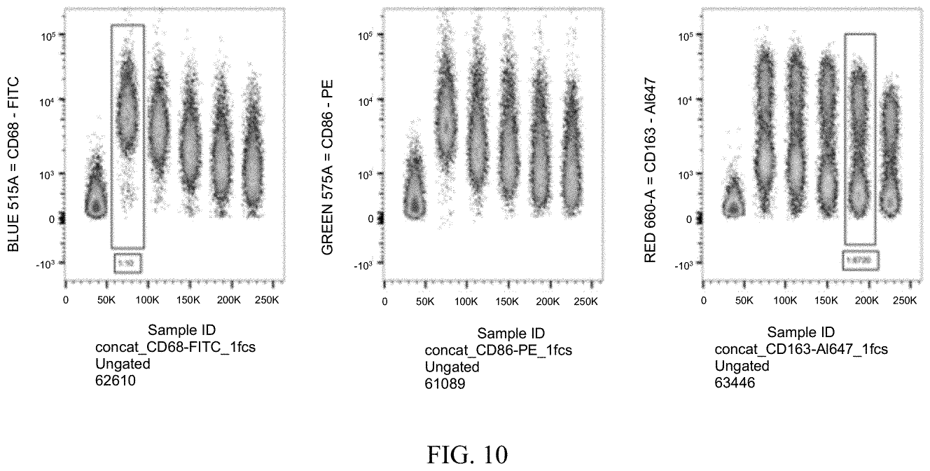

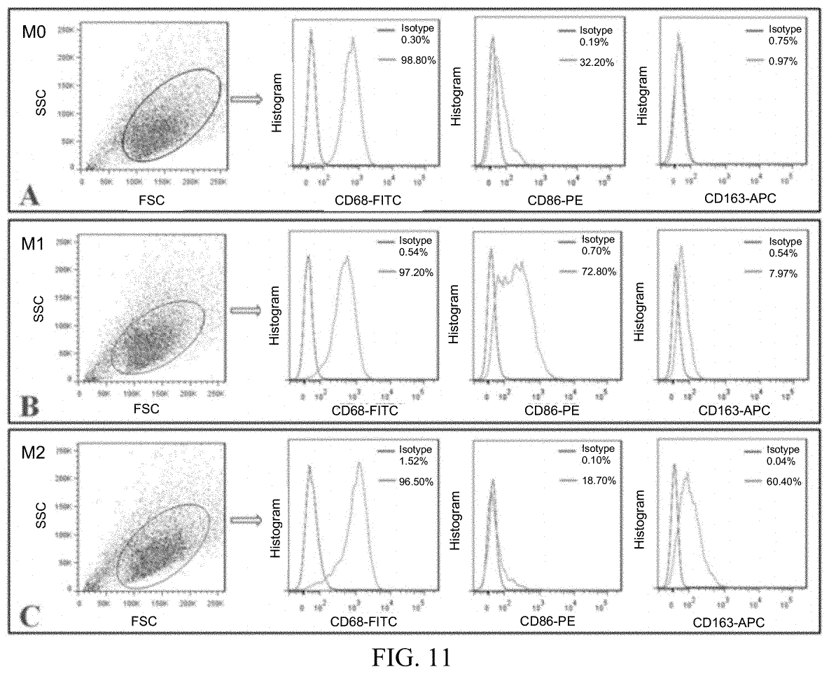

Flow Cytometry: Cells were harvested and washed using TrypLE express (Gibco) and PBS, and centrifugated at 400 g for 5 mins. Cell suspension was adjusted to a concentration of 1-5×10 6 cells in ice cold 0.5 mL Flow Cytometry staining buffer (Invitrogen). Antibodies against cluster of differentiation CD68, CD86, and CD163 were used to identify the macrophage subtypes by flow cytometry. The antibodies was titrated to confirm and identify their optimal expression (FIG. 10). After incubating at room temperature for 1 hour, the flow cytometry was run immediately. For extended storage (16 hr) as well as for greater flexibility in planning time on the cytometer, resuspend cells in 1% paraformaldehyde to prevent deterioration. For immunolabeling, cells were incubated with the following panel of anti-rat monoclonal antibodies: fluorescein isothiocyanate (FITC)-conjugated anti-CD68 (1 μg/10⁶ cells; AbD Serotech, Oxford, UK), Alexa Fluor 647-conjugated anti-CD163 (1 μg/10⁶ cells; AbD Serotech), and PE-conjugated anti-CD86 (0.2 μg/10⁶ cells; eBioscience). 7-AAD (eBioscience) was utilized as a viability staining. After incubation at 4° C. for 30 min, cells were washed three times with staining buffer, fixed with 1% paraformaldehyde (PFA) in PBS and analyzed using BD FACS Aria II (Becton Dickinson, San Diego, Calif.). The non-specific staining was controlled by using isotype-matched antibodies: Mouse IgG1 isotype control FITC (Life Technologies), mouse IgG1 Isotype control Alexa Flour 647 (Invitrogen), mouse IgG1 kappa Isotype Control PE (eBioscience). A minimum of 10,000 events were collected and analyzed by Flow Jo 7.6.1 software (Tree Star, Ashland, Oreg., United States) (FIG. 11).

Cell seeding on collagen scaffolds: A protocol of cells seeding on scaffolds was developed in our laboratory. Briefly, each side of the scaffold was seeded with 25 μL of 250 000 cells, and solutions can stay at the edge of wells (FIG. 8C). The volume of solution was arranged depending on our scaffolds size. The collagen scaffolds were hydrated with %10 FBS for 10 minutes. Following this, the six-well plates were inserted in the incubator for 20 mins to let cells attach and stand. This was followed by the addition of bone marrow growth medium (1:4 of L929 media:10% FBS in DMEM) which scaffolds with cells were cultured overnight at 37° C. and 5% CO2 for 72 hours.

Western Blot and Flow cytometry have been using for identification of macrophages characteristic after seeded on collagen scaffolds.

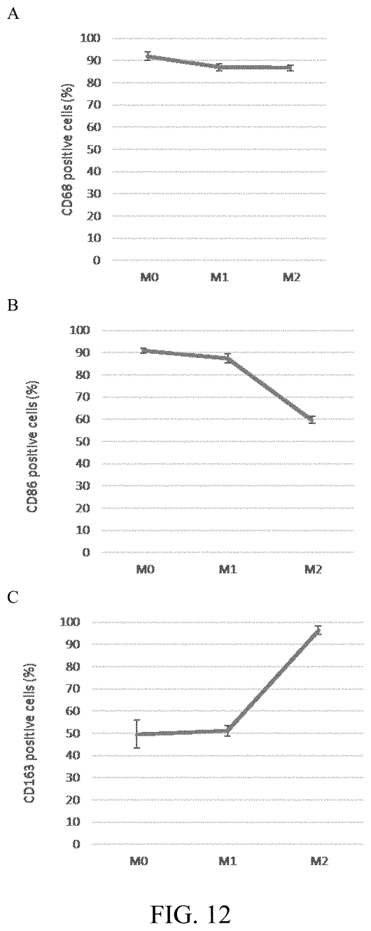

The phenotypes of M0, M1 and M2 cells were identified by Flow Cytometry (FIG. 12). The results showed that nearly all cells were positive for CD68, the marker of macrophages (>75%). CD86 was expressed in most M1 macrophages (72%); the expression was lower in M0 and M2 cells (50.92%). The expression of CD86 in M1 was higher than those in M0 and M2. On the other hand, >90% of M2 cells expressed CD163.

Results

The phenotypes of M0, M1, and M2 cells will be identified by using Flow Cytometry and Western Blot. The results of Western Blot and Flow Cytometry of cells harvested after being seeded on collagen scaffolds are pending.

### Example 4

Polarization of Macrophages from M0 to M2 when Seeded Upon Genipin-Crosslinked Collagen Mesh for Treatment of Stress Urinary Incontinence

Introduction and Objective: The immunological response of the body to implanted biomaterials remains a significant issue. Several important urogynecological applications require tissue support meshes. These scenarios call for materials that promote de novo collagen deposition by the body. A key regulator of collagen production is pro-regenerative M2 macrophages. The creation of advanced biomaterials that encourage monocytes to polarize to M2 macrophages may be an effective way to increase surgical outcomes in tissue reinforcing mesh applications. Our past in vivo study has shown that M2 was greater with genipin implanted group (74). Based on those results, we hypothesized that a genipin-crosslinked collagen mesh will promote polarization to M2. Our aims are: 1) to demonstrate the feasibility of seeding macrophages on genipin-crosslinked mesh for implantation; 2) to illustrate M0 macrophages on the scaffolds undergo a transition to M2 macrophages.

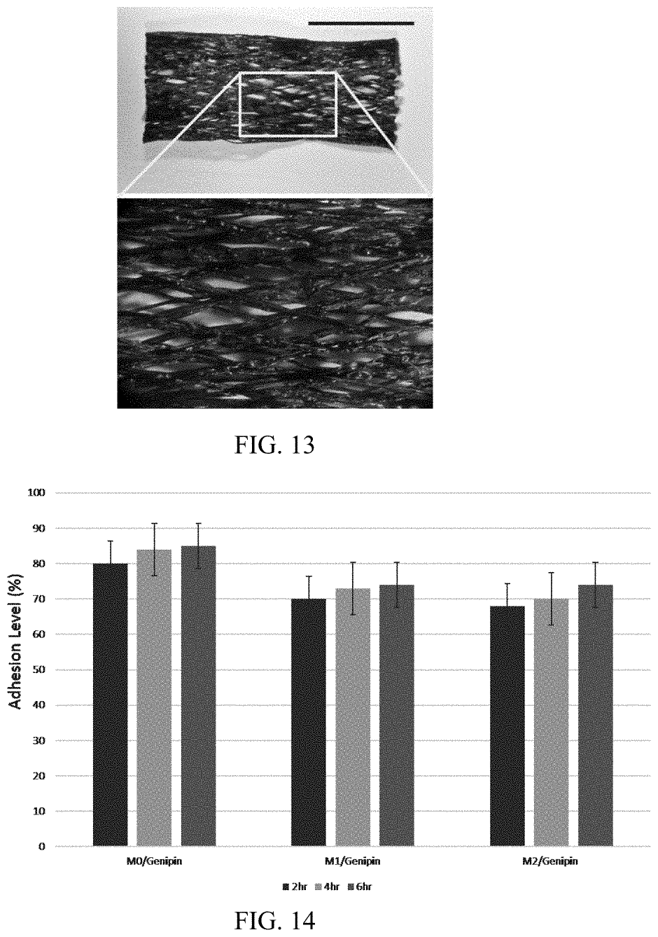

Methods: Monocytes were harvested from rat bone-marrow and macrophage subtypes were identified by using flow cytometry and immunocytochemistry using antibodies against CD68, CD163, CD86, Arginase 1, and iNOS. Electrochemically aligned collagen threads were filament wound to produce small collagen scaffolds, which were then crosslinked using genipin (FIG. 13). Following sterilization, scaffolds were seeded with macrophages and cultured for up to 3 days. Experimental groups included M0 (N=3), M1 (N=3), and M2 (N=3) macrophages. Seeded scaffolds were assessed for cell attachment, proliferation, structure, and protein expression. Western blot for iNOS and Arginase 1 was performed on the macrophages from each group to determine their phenotype at 72 hours.

Results: Adherence of macrophages to genipin crosslinked scaffolds is required for growth of the macrophages and induction of differentiation. Comparisons of percentage adhesion of the macrophages to the scaffolds over time (2 to 6 hours) indicated that macrophages adhered well to the scaffolds (FIG. 14). Macrophages from all groups exhibited over 70% cell attachment at 4 hours following seeding and cell proliferation was similar among all groups.

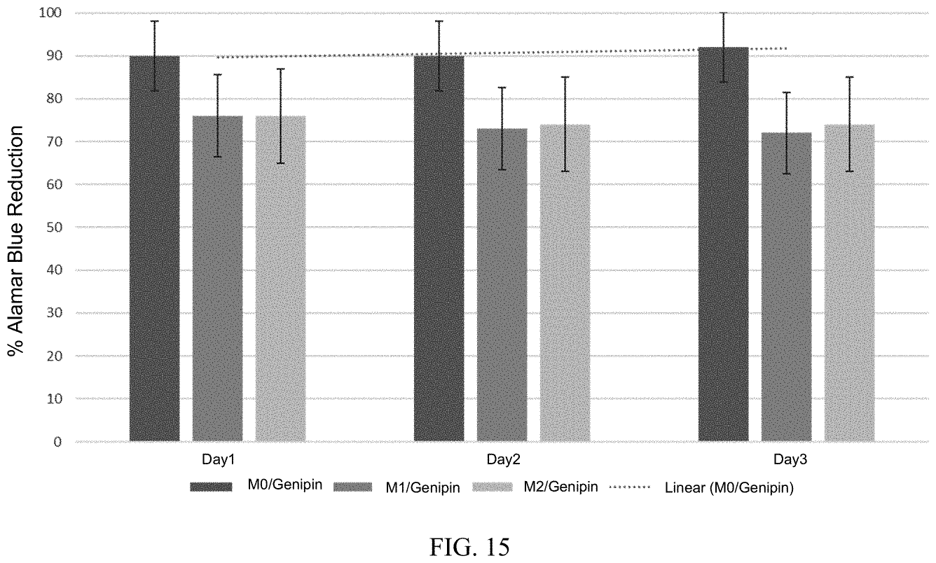

Proliferation of the M0, M1, and M2 macrophages in the genipin crosslinked scaffold preparations was measured over time (1 to 3 days) (FIG. 15). The M0, M1, and M2 macrophages each proliferated well in the scaffold preparations, with the M0 macrophages exhibiting the highest degree of proliferation at each time point.

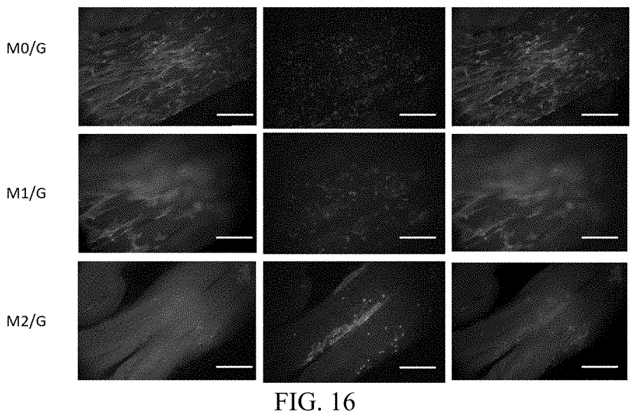

DAPI and phalloidin staining of macrophage-seeded genipin crosslinked collagen scaffolds were also determined (FIG. 16). M0 cells attached well to the scaffolds.

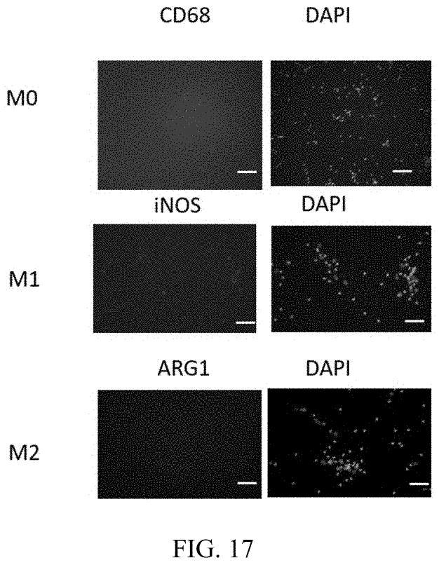

Characteristics of M0, M1, and M2 macrophage as determined by cytochemistry are shown (FIG. 17).

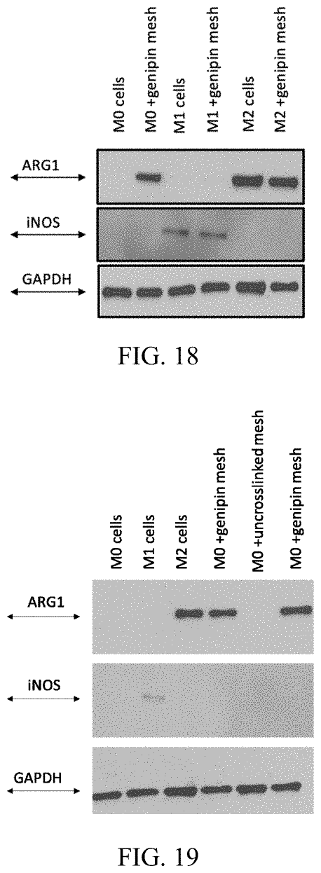

Protein expression highlighted the presence of arginase I and absence of iNOS in M0 cells at 72 hours after seeding on genipin-crosslinked fibers (FIG. 18). Results for macrophages seeded on uncrosslinked mesh and treated with genipin after 72 hours also are shown (FIG. 19).

Conclusions: Genipin-crosslinked collagen scaffolds support sufficient macrophage attachment and survival for implantation. Protein expression results suggest genipin-crosslinked collagen scaffolds induce polarization of M0 macrophages to an M2 phenotype.

### Example 5

Genipin Guides and Sustains the Polarization of Macrophages to the Pro-Regenerative M2 Subtype Via Activation of the pSTAT6-PPAR-Gamma Pathway

Introduction

The fate of biomaterials in vivo is determined by the immunological response to a significant extent. The initial proinflammatory response sets the trajectory of healing as there is prolonged inflammation, fibrosis, and scar tissue deposition around the biomaterial and as well as within the pores of the implant. Leveraging control over the initial phase of inflammatory response may be critical to long-term healing outcomes (75, 76). Blood-borne monocytes are among the first cohort of cells to mitigate tissue damage and interact with a presented foreign material. Depending on physical and chemical characteristics of the biomaterial, monocytes polarize to different forms of macrophages that play a central role in regulating the type and intensity of the host response (77).

Macrophages are immune cells that are categorized as components of the mononuclear phagocyte system, along with monocytes and dendritic cells. Macrophages, depending on their polarization state, may orchestrate accumulative or resolving inflammatory responses. Monocytes generated in bone marrow circulate via the cardiovascular network to supply tissue macrophages whose phenotypes or polarization states depend on alterations in their microenvironment (78). Macrophage polarization is primarily categorized as proinflammatory (M1) and anti-inflammatory (M2), although each category has additional subtypes. It is generally well-accepted that the initial stages of inflammation are predominated by M1 macrophages, and in the longer term, M2 macrophages take over to facilitate wound remodeling (79, 80). Given the significance of macrophage subtypes, numerous important pathways have been acknowledged as essential for polarization such as signal transducers and activators of transcription (STATs), interfering regulatory factors (IRFs), activating proteins (AP1), peroxisome proliferators activating receptors (PPAR-gamma), and cAMP response element binding protein (CREB) (81-82).

M2-macrophages are also known to be pro-regenerative, specifically, they promote deposition of collagen in vivo (83). Establishing advanced biomaterials that promote monocytes to polarize to M2 macrophages may be an effective way to improve surgical outcomes in tissue reinforcing mesh applications in which induction of de novo collagen deposition is essential. Specific load-bearing tissue reinforcement scenarios include repair of hernias, urogynecological tissues, or musculoskeletal soft tissues where accelerated deposition of collagen is required to attain adequate stiffness and strength. However, such a delivery strategy requires a scaffold concept that will: a) enable cell seeding via an interconnected pore volume, b) exhibit baseline mechanical robustness, c) retain seeded cells at the repair site, and most importantly, d) promote and maintain macrophage polarization in the M2 state to deliver the desired pro-regenerative cues (47, 84-85).

Woven biotextiles of electrochemically aligned collagen (ELAC) threads provide mechanical robustness at a highly porous framework that can accommodate cell seeding (1). Previous collagen biotextile assessment in vivo indicated a greater amount of host M2 macrophages were present around genipin crosslinked threads in comparison to carbodiimide crosslinked collagen threads (74). Genipin crosslinked collagen scaffolds were recently reported to promote pro-regenerative macrophage subtype in vitro (85). These observations suggest that genipin may promote M2-polarization; however, it remains unclear whether or not collagen scaffolds also contribute to the picture. Past research demonstrated that cytoskeletal elongation of macrophages via patterned substrates promotes M2-polarization (86). ELAC threads have uniformly aligned collagen molecular deposition which, in turn, induces the elongation of cells topographically. Therefore, the reported elevation of M2-macrophages around ELAC threads in vivo (74) may also have been driven by topographical cues. Therefore, contributions of topography and genipin to M2 polarization need to be resolved. As importantly, identification of pathways by which the polarization occurs can be exploited to regulate polarization process.

We hypothesize that genipin-crosslinked collagen ELAC biotextiles will induce polarization of macrophages from M0 to M2 subtype in the absence of bioinductive cytokines. The aims of the study are: 1) to determine whether M0 macrophages undergo M2 polarization when seeded on genipin crosslinked ELAC threads, 2) determine the role of genipin versus topography induced macrophage cell shape change on the polarization status of cells, 3) to identify the molecular pathway that is implicated in M2 polarization of M0 cells seeded onto genipin-ELAC threads. If genipin-ELAC threads are promotive of M2-polarization state, biotextiles derived from such threads would hold the potential for delivery of pro-regenerative cells to expedite repair in tissue reinforcement applications.

Materials and Methods

1. Scaffold Fabrication

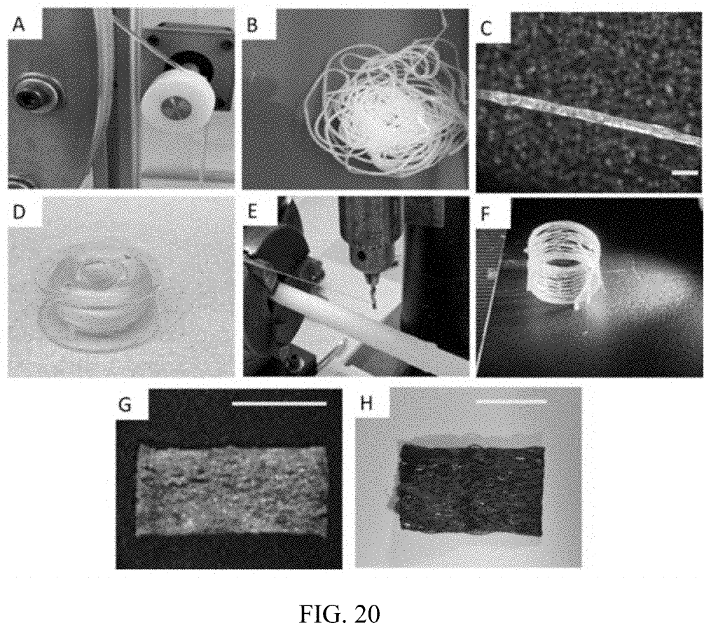

1a. Fabrication of collagen filaments: Electrochemical compaction was used to convert collagen solutions (type I bovine collagen, 3 mg/mL, Collagen Solutions, CA) to aligned threads (˜100 μm) in continuous length on spools as described previously (FIG. 20A) (74). Prior to electrocompaction, collagen solution (3 mg/mL) was dialyzed 24 hours against DI (deionized) water at pH 7.0. The collagen solution was then loaded into a syringe, and it was dispensed in a controlled fashion within the space between the anode and cathode of a rotating electrode wheel. A constant, 40 V of electrical potential was applied between the electrodes. As elucidated earlier, the voltage creates a pH gradient between the electrodes and the resulting charge distribution of ampholytic collagen molecules ends in the net repulsion of molecules by both electrodes. This electrokinetic repulsion compacts and aligns molecules as recoverable monofilament threads in less than 10 seconds of current application. Following electrocompaction, the thread was collected in a solution of 80% isopropanol/20% water (FIG. 20A).

1b. Fabrication of collagen yarns: Individual collagen monofilaments were twisted together manually to form a yarn in order to increase the mechanical strength of the filaments (FIG. 20C). L-ascorbic acid solution (2.7 mg/ml in deionized water) was used to adhere the collagen threads together. The formed yarn was dried in a fume hood, then it was collected on spools and stored at 4° C. (FIG. 20D).

1c. Computer numeric controlled (CNC) filament winding of collagen yarns as scaffolds: A computer numeric controlled machine (Sherline Inc. CA) was used to control the positioning of collagen yarns and overall structure of the collagen scaffolds. The software (LinuxCNC software, v. 2.6.11) was programmed to coordinate motion in two axes: one rotational axis to wind the yarn around a mandrel, and the other a reciprocating linear translational axis along the longer axis of the mandrel (FIG. 20E). Synchronization of speeds of the two axes generated a diamond shaped pore pattern of approximately 5 mm in length and 1 mm in width. During fabrication of scaffolds, the yarns were fused to each other by applying L-ascorbic solution, followed by a drying period after formation of the complete structure. Scaffolds were then recovered from the mandrel (FIG. 20F) and collapsed diametrically to obtain rectangular-shaped scaffolds. Overall dimensions of the scaffold were approximately 20×5×0.5 mm (FIG. 20G).

1d. Genipin crosslinking and sterilization: Collagen scaffolds were crosslinked in genipin solution (2% w/v in 90% ethanol) for 72 h at 37° C. (FIG. 20H). Scaffolds were sterilized prior to further use by treatment for 3 h in peracetic/ethanol solution (1%/22.5% v/v in deionized water).

2. Isolation and Culture of Rat Macrophages

2a. Preparation of Macrophage Growth Medium: L929 cell-conditioned medium is used commonly to support macrophage growth due to secretion of a number of factors by fibroblasts, including Macrophage Colony Stimulating Factor (M-CSF). L929 (NCTC clone 929, ATCC) fibroblasts were cultured in DMEM/F12/GlutaMAX™ (Invitrogen, #10565018) supplemented with L-glutamine 20 mM (ATCC #30-2214), 10% Fetal Bovine Serum (FBS), 1% Penicillin/Streptomycin for 3 days. Bone marrow derived macrophage growth medium was prepared as DMEM/F-12 medium, 10% FBS, 20% L929 cell conditioned medium, and 1% penicillin/streptomycin.

2b. Culture and Polarization of Macrophages: All animal procedures were performed in accordance with rules and protocols approved and established by the Institutional Animal Care and Use Committee (IACUC) at Case Western Reserve University. Bone marrow was harvested from femurs of eight-weeks-old female Sprague-Dawley (SD) rats (220-250 g). Both femurs were immersed in 75% ethanol for 5 min and then left in Dulbecco's phosphate-buffered saline (DPBS) containing 5% Penicillin/Streptomycin. Both ends of bones were cut off using small scissors while holding the diaphysis with forceps. After exposing the intramedullary cavity, a 23 G needle was inserted to inject ice-cold sterile PBS to flush out the marrow into a 15 ml centrifuge tube filled with growth media. The resulting suspension was then passed through a 70 μm cell strainer to remove clumps before centrifuging at 1200 rpm for 5 min to form a cell pellet. The supernatant was discarded and the pellet was resuspended in bone marrow growth medium. Resuspended cells were counted (˜25 million/15 cm dish) and cultured for 7 days in 15 cm2 dishes. Fresh medium was provided every 2 days, cells were passaged before reaching confluence, and these cells were defined as M0 macrophages. For M1 polarization, M0 macrophages underwent a 24 h incubation in 25 ng/ml IFN-γ and 100 ng/ml LPS (Sigma, St. Louis, Mo.). For M2 polarization, M0 macrophages were incubated in 10 ng/ml IL-4, IL-10, and TGF-β1 (R&D systems, Minneapolis, Minn.) for 12 h. Flow cytometry and immunocytochemistry were used to assess macrophage phenotypes.

3. Flow Sorting of Rat Macrophages

Cells were detached using Tryple (Gibco, catalog #12604013), suspended in flow cytometry staining buffer (Invitrogen, catalog #00-4222-57) at 107 cells/mL, and labeled with conjugated antibodies. Cells were stained in polystyrene round bottom 12×75 mm2 Falcon tubes. Primary antibodies against clusters of differentiation CD68, CD86, and CD163 were used to identify the macrophage subtypes by flow cytometry. For immunolabeling, cells were incubated with the following panel of anti-rat monoclonal antibodies: fluorescein isothiocyanate (FITC)-conjugated anti-CD68 (1:10; MCA341F, BioRad, USA), Alexa Fluor 647-conjugated anti-CD163 (1:6720; NBP2-39099, Novus Biologicals, USA), and PE-conjugated anti-CD86 (1:10; 12-0860-83, Thermo Fisher Scientific, USA).7-AAD viability staining (#00-6993-50, eBioscience, USA) was also used for the exclusion of nonviable cells in flow cytometry analysis. After incubation at 4° C. for 30 min, cells were washed three times with a staining buffer and analyzed using a FACSCalibur flow cytometer (Becton Dickinson, San Diego, Calif.). Isotype-matched antibodies, mouse IgG1 kappa Isotype Control PE control (#12-4714-82, eBioscience, USA), mouse IgG1 Isotype Control Alexa Flour 647 (#MA5-18168, Thermo Fisher Scientific, USA) and Mouse IgG1 Isotype Control (#GM4992, Thermo Fisher Scientific, USA), were used to control for non-specific staining that was subtracted from specific staining results. A minimum of 5000 events were collected and analyzed by FCSanalyzer.

4. Immunofluorescence

Macrophages were seeded on glass bottom fluorodishes at 5×104 cells. After induction to different macrophage subtypes, cells were fixed with PBS containing 10% NBF for 10 min, blocked with PBS containing 10% FBS and 0.1% Triton X-100 for 1 hr at room temperature, and then incubated with the follow antibodies: mouse anti-rat CD68 at 1:200 (#sc-20060); mouse anti-rat NOS2 at 1:200 (sc-7271) and mouse anti-rat arginase (#sc-271430) overnight at 4° C. Next day, the samples were incubated at 37° C. for 60 min with conjugated secondary antibodies (1:200) Alexa Fluor 488, Alexa Fluor 555 and Alexa Fluor 595. The samples were rinsed twice in PBS for 5 min and imaged under fluorescent microscopy (Olympus BX50) using water immersion objectives.

5. Phalloidin Staining

The cytoskeletal organization of macrophages cells was assessed using phalloidin staining to visualize F-actin. Samples were rinsed twice in PBS for 3 min and fixed in 10% neutral buffered formalin (NBF) for 15 min. After fixation, they were rinsed twice in PBS for 5 min. Samples were permeabilized with 0.1% Triton X-100 in PBS for 15 min, then rinsed twice in PBS for 3 min. Samples were then stained with Alexa 488-conjugated phalloidin (Cat #A12379 Thermo Fisher Scientific) for 30 min while protected from light. Samples were rinsed twice in PBS for 5 min and imaged under fluorescent microscopy (Olympus BX50) using water immersion objectives. Cell area measurements were conducted by using Image J software analysis. The degree of cell elongation was calculated as the length of the longest axis divided by the length of the shortest axis of cytoskeleton.

6. Cell Proliferation Assay

AlamarBlue assay was conducted on days 1, 2, and 3 to quantify the proliferation of M0, M1 and M2 on the scaffolds by measuring the metabolic activity of the living cells. The media was removed and 100-4, AlamarBlue reagent (DAL1025, Fisher Scientific) was added to each 96-well plate. The plates were incubated for 4 h at 37° C. and absorbance at 595 nm was measured using a microplate reader (SpectraMax M2, USA). Three replicates for each group of samples and controls were included for repeatability.

7. Western Blotting

7a. Effects of genipin crosslinked scaffolds on polarization status of M0, ML and M2 macrophages: We sought to investigate the changes in the polarization status of M0, M1, and M2 macrophages following seeding onto genipin crosslinked ELAC scaffolds. Scaffolds were seeded with prepolarized M0, M1, or M2 macrophage subtypes and cultured for up to 3 days (N=3/group). There were no additional cytokines in the culture medium of cells seeded on scaffolds to ensure that effects on polarization were limited to the scaffold material. Macrophage subtypes cultured on tissue culture plastic were used as positive controls. Flow-sorted, polarized macrophages were seeded at a density of 2×10⁵ cells per scaffold by addition of 50 μl cell suspensions at a concentration of 4 million cells/ml. Cells on scaffolds were then incubated for 30 min at 37° C. and 5% CO2 to allow for attachment. Two ml of culture media were added to each well on the 6-well cell culture plates.

7b. Effects of genipin and cellular elongation on M0 macrophages: The experimental design elucidated in section 7a does not differentiate between the effects of collagen alignment induced cellular elongation and the effects of genipin on macrophage subtype polarization. A second set of experiments included the following groups: 1) Tissue culture plate with M0-polarization medium, 2) Tissue culture plate with M1-polarization medium, 3) Tissue culture plate with M2-polarization medium, 4) Tissue culture plate with M0-polarization medium with direct addition of genipin, 5) M0 subtype cells seeded on uncrosslinked collagen threads, 6) M0 subtype cells seeded on genipin crosslinked collagen threads. In this design, Groups 1-3 constitute the positive controls. Comparison of group 1 with group 4 reveals the effects of genipin in soluble form on M0 subtype polarization. Comparison of group 1 with group 5 reveals the effect of aligned collagen topography on M0 subtype. Comparison of group 5 with group 6 determines how genipin crosslinking of collagen affect the polarization status of M0 subtype. For group 4, genipin (Wako Pure Chemical Industries, catalog #078-03021, Richmond, Va.) in 0.1% dimethyl sulfoxide (DMSO, Sigma-Aldrich, USA) (12.5 μg/ml, genipin/DMSO) was directly added to the culture medium. This concentration was determined based on a previously reported data that demonstrated this level to be subtoxic (87).

7c. Investigation of signaling pathways involved in M2 polarization by genipin: A third set of experiments included the following groups: 1) Tissue culture plate with M2-polarization medium, 2) Tissue culture plate with M0-polarization medium with direct addition of genipin, 3) Tissue culture plate with M0-polarization medium. In this design, Groups 1 and 3 represent the positive and negative controls.

Total cell lysate was prepared using RIPA lysis buffer (#50195822), phenylmethylsulfonylfluoride (PMSF) (#NC0417414) and protease/phosphatase inhibitor (#50193743) purchased from Cell Signaling Technology. The protein concentration in the cell lysate was estimated using DC protein assay (BioRad) and 25 μg protein was resolved using 4-20% Mini-PROTEAN gels (#4561094, BioRad) and transferred to a nitrocellulose membrane (#1620213, BioRad). The membrane was blocked in 5% non-fat dry milk/1% Tween 20 for 2 hr at room temperature. They were incubated with primary antibodies anti-Arg1 for M2 (sc-271430), anti-iNOS for M1 (sc-7271), pSTAT6 (#9361S), PPAR-gamma (sc-7273) and anti-GAPDH (sc-365062) as the housekeeping protein overnight at 4° C. Following day, the blots were washed in TBST and incubated with anti-mouse secondary antibody (sc-516102) conjugated with horseradish peroxidase detected by ECL-chemiluminescence, then underwent autoradiography using BioBlue-Lite™ film (Alkali Scientific, FL). Quantitative analysis of the protein bands was performed using the Image J.

8. Statistical Analysis

All statistical analyses in this study were performed using GraphPad Prism 7.00 (GraphPad, Inc., La Jolla, Calif., USA). P values of <0.05 were considered significant and denoted with stars. All reported results are expressed in terms of the mean±standard deviation. Mann-Whitney U-test was used for all analyses.

Results

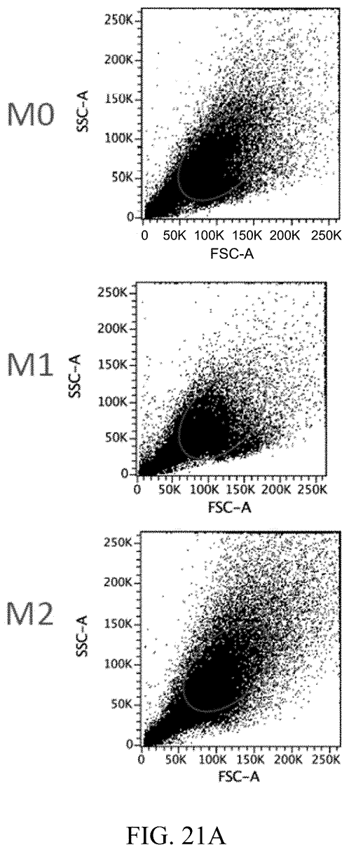

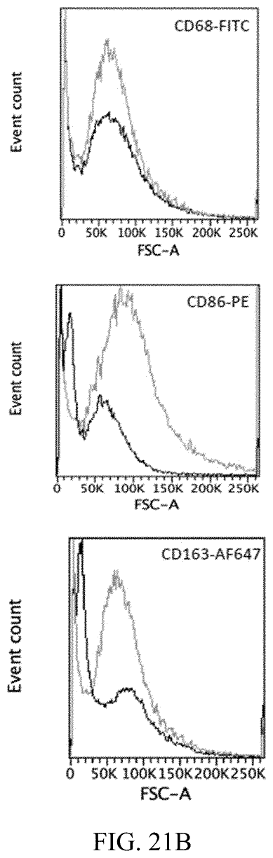

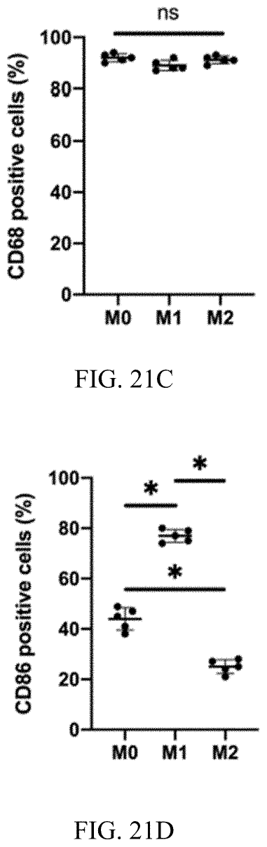

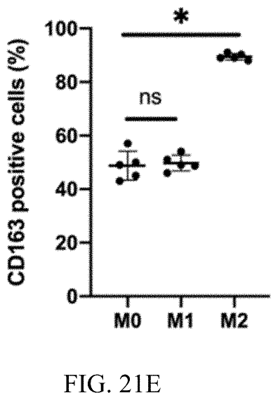

Cell populations of interest were gated successfully (FIG. 21A) and corresponding matched isotype control staining histograms were generated (FIG. 21B). The results demonstrated that almost all macrophages (M0, M1, and M2) were positive for the pan-macrophage marker CD68, as expected (FIG. 21C) (88-89). CD86 expression for M1 macrophages was significantly higher than those of M0 and M2 macrophages (p<0.05, FIG. 21D). M1-marker CD86 was present at the lowest level for macrophages that were cultured under M2-polarization conditions. CD163 was expressed in macrophages that were cultured under M2-polarization conditions more than those cultured under conditions promoting M0 and M1 phenotypes (p<0.05, FIG. 21E). Overall, flow cytometry confirmed induction of desired phenotype was successful to a significant extent.

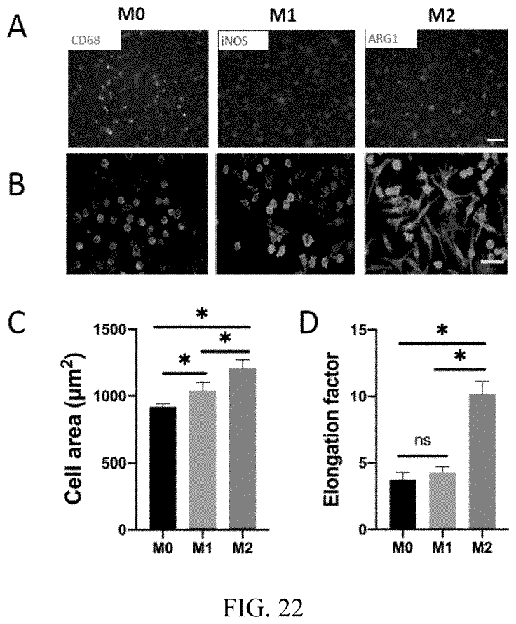

The phenotypes of polarized macrophages were also confirmed by immunofluorescence where populations of cells cultured under M0, M1, and M2 polarization conditions were positive for CD68 (green), iNOS (red), and arginase 1 (orange-red), respectively (FIG. 22A). Based on confirmation of phenotypes via flow cytometry and immunofluorescence, these populations will be referred to as M0, M1, and M2 macrophages hereon in this example.

Phalloidin-stained images demonstrated bone marrow-derived macrophages seeded on tissue culture plastic and stimulated with cytokines to induce M1 or M2 polarization displayed markedly different cytoskeletal morphologies. Unpolarized M0-cells were round in shape (FIG. 22B). Addition of LPS and IFN-γ, which stimulate M1 polarization, caused cells to spread over a larger area (FIG. 22C) than M0 cells while maintaining a characteristic round morphology (FIG. 22D). In contrast, addition of IL-4/10 and TGF-β, which stimulate M2 polarization, led to a spindle-like cellular morphology (FIG. 22B). M2 cells exhibited a significantly higher cell area and degree of elongation compared to M0 and M1 cells (p<0.05, FIG. 22C&D).

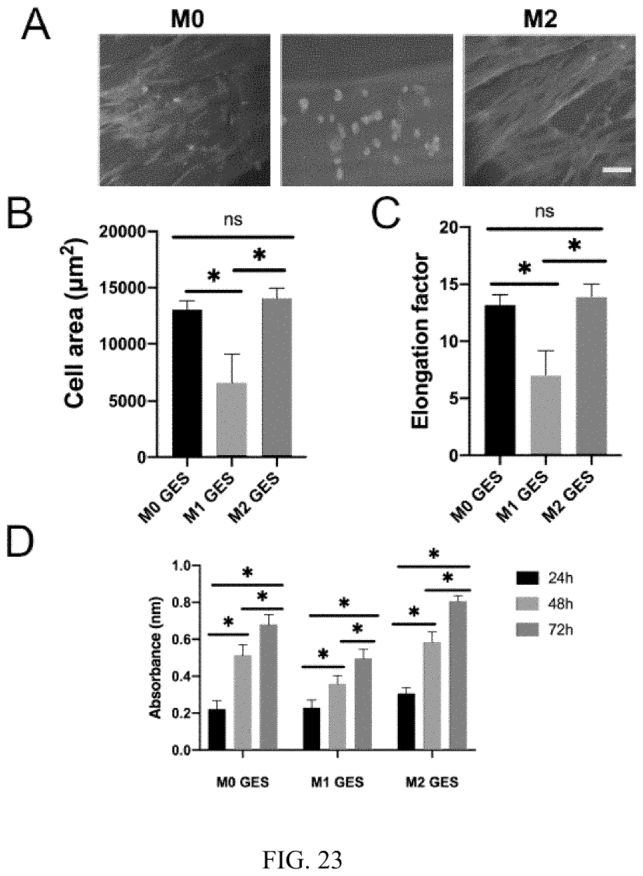

Cytoskeletal morphologies of polarized macrophages seeded on aligned genipin crosslinked collagen threads were visualized by phalloidin staining. M0 and M2 macrophages demonstrated a significant degree of elongation as well as uniform alignment along the longer axis of genipin crosslinked threads (FIG. 23A). In contrast, the cytoskeletal structures of M1 cells remained round in shape (FIG. 23A). M2 and M0 polarized cells had significantly greater cell areas and also significantly more elongated than M1 cells (p<0.05, FIG. 23B&C). There were no significant differences between cell area or elongation of M0 and M2 polarized cells (FIG. 23B-C). Elongation factors for M0/M2 cells on seeded on ELAC threads were greater than those of M0/M2 cells cultured on tissue plate (FIG. 23C vs FIG. 22B, respectively), implying that collagen threads imparted additional elongation to these phenotypes.

Cell proliferation of polarized macrophages on genipin crosslinked collagen scaffolds was inferred by metabolic activity measurements at 24, 48, and 72 h. As illustrated (FIG. 23D), increasing absorbance was detected for each macrophage subtype such that absorbance at each time point was significantly greater than the prior time point (p<0.05, FIG. 23D). It was also observed that M1-subtype proliferated at a slower rate than other sub-types.

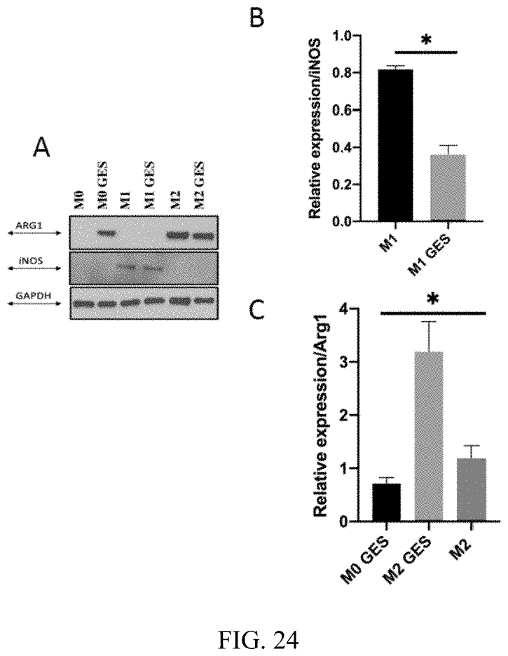

We examined whether or not prepolarized M0, M1, and M2 macrophages sustain their polarization status after being seeded on genipin crosslinked ELAC scaffolds in the absence of cytokines in the medium. iNOS (M1 marker) and arginase-1 (M2 marker) expressions were evaluated by Western Blotting. Protein expression highlighted the presence of arginase I and the absence of iNOS in M0 cells at 72 hours after seeding on genipin-crosslinked fibers (M0 vs. M0-GES in FIG. 24A), indicating emergence of M2 sub-types. M2 subtype expression was not only maintained on genipin crosslinked ELAC scaffolds (M2 vs. M2-GES, FIG. 24A) but also at greater levels than in M2 cells in tissue culture plastic (M2 vs. M2-GES in FIG. 5C), indicating that genipin crosslinked collagen threads spike M2 expression. Unlike M0 and M2 subtypes, arginase-1 expression was absent in M1 subtype when seeded on genipin crosslinked ELAC threads; however, a reduction in iNOS expression for seeded M1 macrophages was noted (M1 vs M1-GES, p<0.05, FIG. 24B).

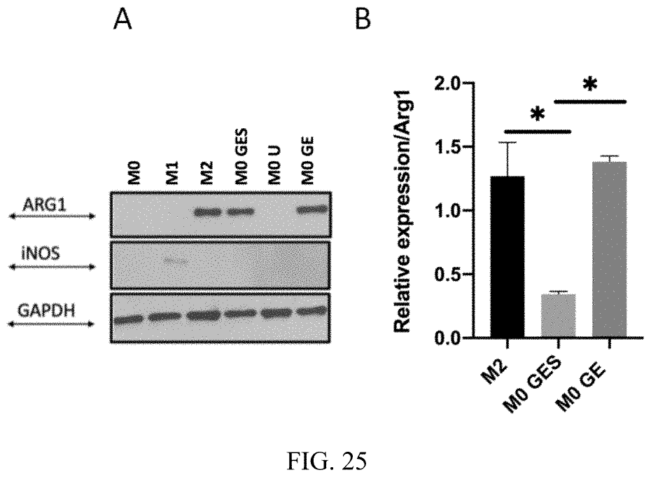

To explore the individual roles of genipin and ELAC on the induction of M2 polarization reported in FIG. 24, M0 macrophages were seeded on ELAC threads (crosslinked: M0-GES and uncrosslinked: M0-U), as well as a separate group of M0 cells were seeded on tissue culture plate and treated by direct addition of genipin to the culture media (M0-GE). iNOS and arginase-1 markers were evaluated by Western blotting (FIG. 25A). Quantitative analysis showed that arginase-1 expression in genipin treated group M0-GE was higher than for the M2 subtype induced with standard cytokines (FIG. 25B). M0 cells seeded on genipin crosslinked threads expressed arginase-1, whereas those seeded on uncrosslinked collagen threads did not express arginase-1. Taken together, our data show that M2-subtype conducive effects of genipin crosslinked ELAC scaffolds emerge from genipin and not from the aligned collagen induced cellular elongation.

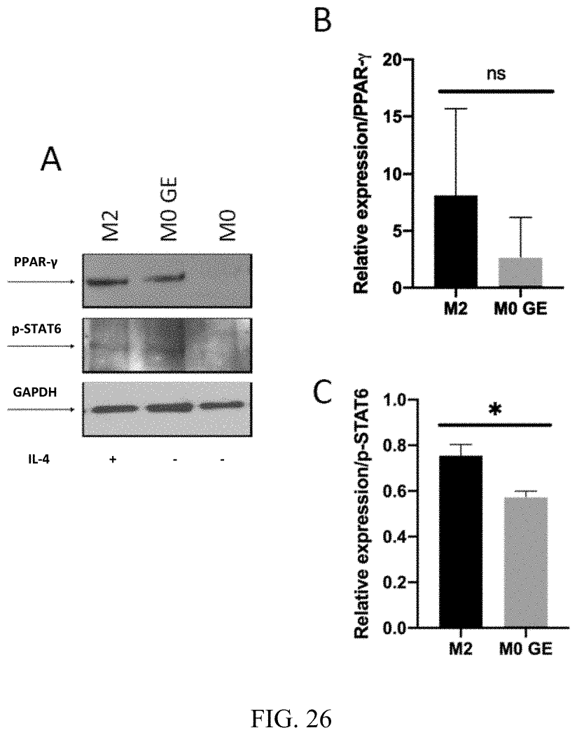

To explore which signaling pathways are involved in genipin induced M2 polarization, the M2 classical pathway proteins (pSTAT6 and PPAR-γ) were evaluated by Western Blotting (FIG. 26A). Protein analysis exhibited that genipin treated M0 group expressed pSTAT6 and PPAR-γ. Quantitative analysis showed that PPAR-gamma expression in genipin treated group M0-GE was similar to the M2 subtype induced with IL-4 (FIG. 26B). However, pSTAT6 expression was significantly higher compared to M2 polarization induced by IL-4 (FIG. 26C). Taken together, our data show that the pSTAT6-PPAR-γ signaling pathway to be involved during genipin induced M2 polarization.

Discussion

Collagen biotextiles fabricated by weaving electrochemically aligned threads are an effective vehicle to deliver cells owing to the receptiveness of collagen for cell adherence and the presence of a macroscale, connected pore network that facilitates cell seeding (1, 90). Therefore, in tissue reinforcement applications ELAC scaffolds can deliver macrophages of a pro-regenerative sub-type to promote deposition of connective tissue. The main aim of this study was to determine whether genipin crosslinked, aligned collagen threads support cells with an M2 macrophage subtype. The effects of the biomaterial on M0 and M1 subtypes were also investigated. We showed that the pro-regenerative M2-subtype was enhanced and the pro-inflammatory M1-subtype was dampened by the biomaterial. Importantly, genipin crosslinked collagen threads polarized M0 subtype to M2 subtype. All these effects were observed to occur in the presence of the biomaterial alone, in other words, absent were any exogenous cytokine or other bioinductive factors in the medium. The second aim of the study resolved the roles of genipin and collagen induced cytoskeletal alignment on the reported macrophage polarization. It was demonstrated by direct addition of genipin to M0 subtype culture, again in the absence of inductive factors, that the M2-polarizing effect of genipin crosslinked collagen threads was reliant on genipin and not on collagen induced alignment. In this regard, the study underlines the influence of the genipin crosslinked collagen scaffold on the regenerative response and highlights the implication of understanding multifaceted interactions of macrophage responses.

Previously, it was demonstrated that ELAC threads elongate MSCs which in turn induces tenogenic differentiation as a result of topographical cues (1, 55). In this study, we observe a similar elongation inducing effect of aligned collagen threads on cytoskeletons of M0 and M2 macrophage subtypes. A role for cell elongation in the regulation of macrophage phenotype polarization was reported before (86) such that the elongation of macrophages led to higher levels of M2-subtype markers arginase-1, CD206, and YM-1. We did not see a similar effect of elongation by itself inducing M0 to M2 polarization as indicated by the absence of Arg1 expression in the group of cells that were seeded on uncrosslinked, aligned collagen threads (MOU, FIG. 25). This discrepancy may result from several differences in study designs where the former study used fibronectin and the current study used collagen, which may involve different integrin units and could trigger different differentiation pathways. Furthermore, the former study relied on microstamped grooves to obtain a higher degree of elongation than observed in our study. It is also noteworthy that the M2 induction capacity of elongation is substantially milder (4-fold increase in Arg1) than when induced by soluble cues (IL4/IL10/TGF-β, 120-fold increase in Arg1). These differences may explain why elongation was not a dominant factor in the upregulation of M2 polarization reported in the current study. However, matrix induced elongation maintained M2 polarization status, such that M2 cells seeded on aligned scaffolds had greater Arg1 expression than M2 cells on culture plates (M2GES vs. M2, FIG. 24) despite the absence of exogenous cytokines in the culture medium for the former group. This outcome indicates that once cells are induced to an M2 form in a culture plate and seeded on ELAC threads, the phenotype will not only be maintained but further enhanced. In addition to cellular elongation, there is recent evidence that collagen fibril density drives anti-inflammatory traits in THP-1 monocytes when embedded in three-dimensional (3D) collagen matrices (91). Even if not directly, scaffolds may augment the polarization of macrophages as these studies indicate collectively.

It was observed that once differentiated to the M1-subtype, cell maintained a round cytoskeletal shape and resisted elongation on threads, unlike M0 and M2 macrophages. This result suggests that cytoskeletal pathways are terminally set in M1 macrophages and they may be less plastic in terms of repolarization. While there are reports of M2 subtype being inducible to M1 subtype (i.e. repolarization) (92, 93), it is reported that repolarization of M1 to M2 subtype is not as feasible (94). Differential response of cytoskeletal plasticity for M1 vs. M2 will be a matter of future research where cytoskeletal pathways such as Cdc42, Rho and Rac would be investigated.

Genipin has been successfully used for crosslinking collagen-based materials such as biological tissues and other natural polymers (95). We have demonstrated earlier that collagen thread's mechanical robustness can be adjusted to match that of strong native tissues, such as tendon, using genipin as the chemical crosslinking agent (55). Genipin has traditionally been used in traditional medicine in Asia for centuries and is reported to have anti-inflammatory properties (48-49, 96-97). Several studies have shown that the genipin scaffolds elicited a predominantly M2 phenotype response in vivo (74, 98). However, there are limited controlled in vitro studies to resolve the specific effects of genipin on macrophage polarization. We observed an increase in pro-regenerative M2/Arg1 response when M0 macrophages were treated with genipin by direct addition to the culture medium, as well as when they were seeded on genipin crosslinked collagen scaffolds. However, addition of soluble genipin exhibited an approximately 3-fold greater increase in Arg1 expression as compared to genipin presented via crosslinked collagen (MOGES vs. MOGE, FIG. 25B). Importantly, M2 inductive capacity of soluble genipin addition was moderately greater than a potent cytokine (IL4/IL10/TGF-β) combination (M2 vs. MOGE, FIG. 24B). Several studies have shown that genipin crosslinked collagen diminishes a proinflammatory response while enhancing an anti-inflammatory response from macrophages, which are supportive of our findings (85, 99). Different from these studies, we applied genipin directly to the solution and also in the absence of other cytokines to get a definitive answer on genipin's inductive capacity. Furthermore, we have shown maintenance of the M2-polarization state, M0→M2 polarization induction, along with suppressed proinflammatory M1/iNOS response after being seeded on genipin ELAC scaffolds. Together with multiple reports in the literature, these results strongly suggest that genipin crosslinking directs macrophage polarization away from a pro-inflammatory phenotype and toward a pro-regenerative phenotype.

Previously it was reported that genipin has anti-inflammatory and anti-angiogenic effects (49). Mechanisms by which genipin induces pro-regenerative polarization of macrophages are not yet fully elucidated. For most cell types, genipin is reported to suppress pro-inflammatory markers; such as TNF-α and IL-6, produced from LPS-stimulated RAW 264.7 murine macrophages cell line (96) and TNF-α-induced VCAM-1 expression in human endothelial cells (100). Other cell types may behave differently, for example, genipin treatment leads to IL-10 and TNF-α production from LPS-stimulated microglia cells derived from rat brain (97). These reports suggest that while genipin typically inhibits some inflammatory factors, including cytokines and adhesion molecules, its anti-inflammatory effect on a given cell type is not guaranteed and should be assessed prior to proceeding beyond initial experiments. In this study, we identified p-STAT6-PPAR-γ molecular pathway's involvement in M2 polarization using western blot analysis in vitro. STAT6 is the crucial transcription factor in IL-4 mediated M2 polarization (101, 102). Peroxisome proliferator-activated receptor-γ is a member of the nuclear receptor superfamily of ligand-dependent transcription factors that primarily inhibit M1 polarization in adipose tissue and adrenal glands. It is expressed moderately in macrophages, but its expression can also be prompted by IL-4 and IL-13, which shows a probable role for the nuclear factor in M2 polarization (103, 104). STAT6 is known as a cofactor in PPAR-γ mediated gene regulation in vitro; hence, crosstalk between PPAR-γ and IL-4-STAT6 axis directs the M2 phenotype. PPAR-γ deficiency results in downregulation of M2 polarization and development of obesity, insulin resistance and hepatic steatosis (105).

It has been well accepted that macrophages are heterogeneous and undergo phenotypic changes/functional switches, depending on microenvironmental stimuli (79, 106). Based on a large amount of literature in the relevant field of macrophage polarization, we selected the marker of M1 polarization-iNOS and M2 polarization-Arg-1. iNOS in macrophages is linked to M1, whereas ornithine generated from arginase is associated with M2 phenotype (107). A considerable number of studies determined that Arg-1 is dominantly expressed in M2 cells and reduces NO production from iNOS through limiting bioavailability of intracellular 1-arginine, resulting in diminished inflammatory tissue damage and suppression of intracellular pathogen clearance (108-110).

There were several limitations to the current study. While we verified the polarization status of macrophages in defined inductive media extensively by using western blot, flow cytometry, and immunofluorescent analyses; the polarization status in response to scaffold effects was confirmed by using protein expression alone. While western blot analyses are more direct than gene-expression (111), elucidation of growth factor secretion to the culture medium to assess macrophage functional response to genipin will be necessary for future studies. In particular, treatment of fibroblasts with culture media conditioned by macrophages polarized at different states would confirm the functional output of macrophages. Despite these limitations, this study shows that the genipin crosslinked ELAC scaffold is a promising delivery vehicle to present macrophages to repair sites in a pro-regenerative format.

In terms of a future direction, we propose that blood-derived monocytes from patients can be applied directly to genipin crosslinked ELAC scaffolds and implanted in the repair site. Release of pro-regenerative factors by M2-polarized macrophages, such as TGF-beta family proteins that drive de-novo collagen production, may help address clinical applications requiring structural augmentation. Applications such as hernia, orthopedic soft tissue repair, or urogynecological repair for stress urinary incontinence or pelvic prolapse could benefit significantly from this approach.

Conclusions

Taken together, results from this study highlight the importance of understanding the macrophage response to genipin crosslinked collagen scaffolds in order to positively influence the regenerative response after biomaterial implantation. In this vein, studies are in progress currently to assess the biological responses and biomechanics of repairs in and around M0, M1 and M2 polarized ELAC biotextiles implanted in vivo.

### Example 6

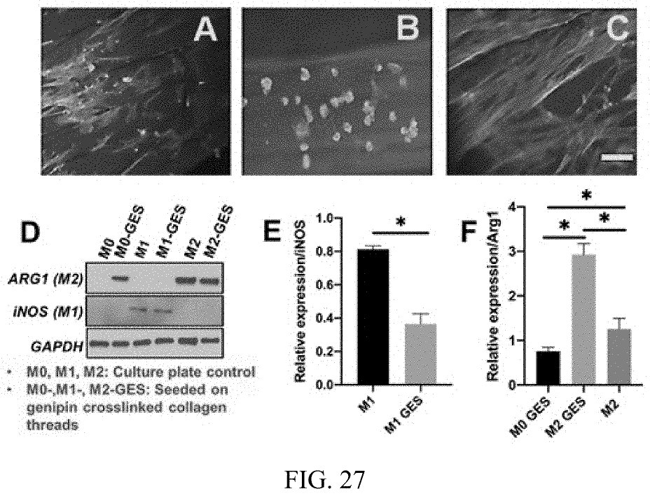

Genipin crosslinked collagen biotextile maintains M2 macrophage polarization in vitro: Depolarization of M2 macrophages to a different subtype due to the composition of a biotextile would not be desirable. Accordingly, we sought to determine whether polarization status of M1 and M2 subtypes are affected by genipin crosslinked collagen threads. Rat monocytes collected from bone marrow were polarized to M0 (unpolarized), M1 and M2 subtypes, and seeded on genipin-collagen threads (M0-GES, M1-GES and M2-GES). Tissue culture plates with inductive media specific to each subtype were prepared in parallel as positive controls (M0, M1, M2). There were no cytokines in the culture media of cells seeded on scaffolds to ensure that effects on polarization were limited to those imparted by the scaffold material. Cell extracts were prepared on day 3 to measure iNOS (M1-marker) and Arg1 (M2-marker) protein expressions by Western-blot, with GAPDH as the housekeeping control. Morphological visualization of cytoskeletal actin indicated that all macrophage groups were adherent to collagen threads (FIG. 27A-C) which indicates these threads are suitable for in vivo delivery. Western blot results indicated that genipin crosslinked CollaMesh do not depolarize M1 (iNOS present and Arg 1 absent both in culture plate and on threads, FIG. 27D-F) or M2-subtypes (Arg1 present and iNOS absent both in culture plate and on threads, FIG. 27D-F), and enhance M2-traits in M0 subtypes. Maintaining baseline polarization status of M1 and M2 cells as such renders genipin CollaMesh suitable for delivery of macrophages for immunoregenerative purposes.

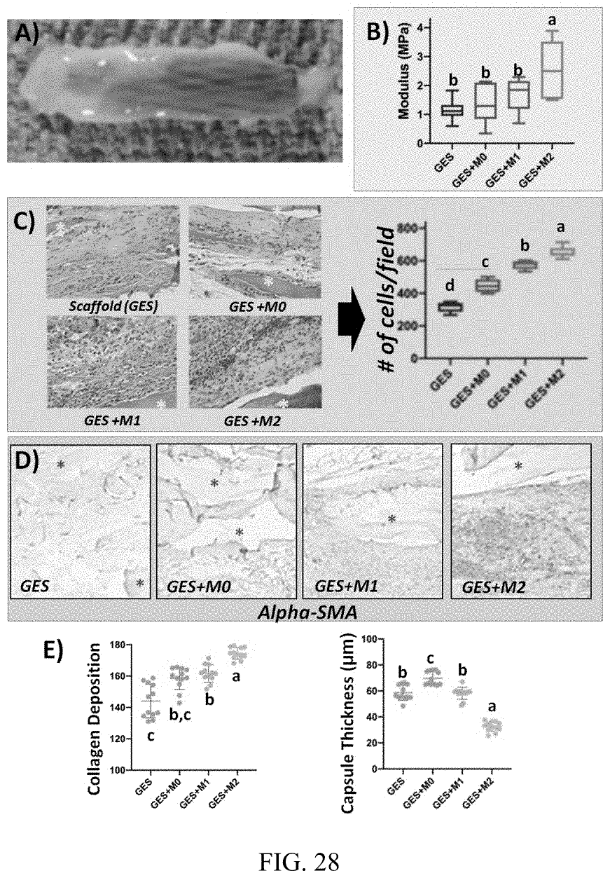

In vivo delivery of M2-subtype by filament wound collagen scaffolds results in superior healing biomechanics and implant remodeling in comparison to other macrophage subtypes: M0, M1 or M2 macrophages were polarized from monocytes, seeded on collagen textile and implanted subcutaneously in rats to determine whether macrophage supplementation improves the stiffness (i.e. modulus) of regenerated tissue, collagen deposition by host, and alters the biology of healing. Implants were recovered at 3 months (FIG. 28A) following which they were tested in tension, cellularity was quantified in HE stained sections, collagen production was quantified by automated image analysis of Masson's trichrome sections, and fibrous capsule thickness was measured. M2-subtype seeded scaffolds (GES+M2) had the greatest modulus such that their magnitude was about 2.5 times greater than that of cell-free scaffolds (GES) (FIG. 28B). Moduli of GES+M0 or GES+M1 scaffolds did not differ significantly from that of cell-free scaffolds. Number-of-cells count per unit area was the greatest for GES+M2 group, suggesting a greater degree of host cell attraction (FIG. 28C). Furthermore, alpha smooth muscle actin, a marker of myofibroblasts, was more notably present in M2-seeded scaffolds (FIG. 28D). M2 delivery induced a greater amount of collagen deposition while limiting fibrous encapsulation of the scaffold (FIG. 28E). The net effect of M2-delivery was improved healing biomechanics that converged closer to the 3-5 MPa native tissue modulus range. The scaffold alone is unable to converge. Attaining better modulus with a diminished level of capsule implies that the quality and quantity of tissue deposition within the scaffold was improved by the mediation of M2 subtype.

Conclusion: These results provide evidence that macrophage supplementation, particularly M2-subtype, benefit the regeneration.

### LITERATURE CITED

1. Younesi, M., Islam, A., Kishore, V. et al.: Tenogenic Induction of Human MSCs by Anisotropically Aligned Collagen Biotextiles. Advanced Functional Materials, 24: 5762, 2014.
2. Jonsson Funk, M., Levin, P. J., Wu, J. M.: Trends in the surgical management of stress urinary incontinence. Obstet Gynecol, 119: 845, 2012.
3. Shah, H. N., Badlani, G. H.: Mesh complications in female pelvic floor reconstructive surgery and their management: A systematic review. Indian J Urol, 28: 129, 2012.
4. Seklehner, S., Laudano, M. A., Xie, D. et al.: A meta-analysis of the performance of retropubic mid urethral slings versus transobturator mid urethral slings. J Urol, 193: 909, 2015.
5. Fatton, B., de Tayrac, R., Costa, P.: Stress urinary incontinence and LUTS in women—effects on sexual function. Nat Rev Urol, 11: 565, 2014.
6. Davila, G. W., Drutz, H., Deprest, J.: Clinical implications of the biology of grafts: conclusions of the 2005 IUGA Grafts Roundtable. Int Urogynecol J Pelvic Floor Dysfunct, 17 Suppl 1: S51, 2006.
7. Milose, J. C., Sharp, K. M., He, C. et al.: Success of Autologous Pubovaginal Sling Following Failed Synthetic Midurethral Sling. J Urol, 2014.
8. FitzGerald, M. P., Mollenhauer, J., Brubaker, L.: The fate of rectus fascia suburethral slings. Am J Obstet Gynecol, 183: 964, 2000.
9. Chapin, K. J., Khalifa, A., Anderson, J. M. et al.: In vivo tissue integration and healing stiffness of woven collagen meshes: comparison to porcine dermis and polypropylene.
10. Khalifa, A., Chapin, K., Islam, A. et al.: MESH WOVEN FROM PURE COLLAGEN THREADS FOR TREATMENT OF STRESS URINARY INCONTINENCE. Presented at the NEUROUROLOGY AND URODYNAMICS, 2016.
11. Herrera-Imbroda, B., Lara, M. F., Izeta, A. et al.: Stress urinary incontinence animal models as a tool to study cell-based regenerative therapies targeting the urethral sphincter. Adv Drug Deliv Rev, 2014.
12. Norton, P., Brubaker, L.: Urinary incontinence in women. Lancet, 367: 57, 2006.
13. Norton, P., Brubaker, L., Nager, C. W. et al.: Pelvic organ prolapse in a cohort of women treated for stress urinary incontinence. Am J Obstet Gynecol, 211: 550 e 1, 2014.
14. Buckley, B. S., Lapitan, M. C.: Prevalence of urinary incontinence in men, women, and children—current evidence: findings of the Fourth International Consultation on Incontinence. Urology, 76: 265, 2010.
15. Resnick, N. M., Griffiths, D. J.: Expanding treatment options for stress urinary incontinence in women. JAMA, 290: 395, 2003.
16. MediPoint: Women's Health—Global Analysis and Market Forecasts: GlobalData, 2015.
17. Cosson, M., Debodinance, P., Boukerrou, M. et al.: Mechanical properties of synthetic implants used in the repair of prolapse and urinary incontinence in women: which is the ideal material? Int Urogynecol J Pelvic Floor Dysfunct, 14: 169, 2003.
18. Babcock, W. W.: The range of usefulness of commercial stainless steel clothes in general and special forms of surgical practice. Ann West Med Surg, 6: 15, 1952.
19. Petros, P.: Creating a gold standard surgical device: scientific discoveries leading to TVT and beyond: Ulf Ulmsten Memorial Lecture 2014. Int Urogynecol J, 26: 471, 2015.
20. Zheng, F., Lin, Y., Verbeken, E. et al.: Host response after reconstruction of abdominal wall defects with porcine dermal collagen in a rat model. Am J Obstet Gynecol, 191: 1961, 2004.
21. Dora, C. D., Dimarco, D. S., Zobitz, M. E. et al.: Time dependent variations in biomechanical properties of cadaveric fascia, porcine dermis, porcine small intestine submucosa, polypropylene mesh and autologous fascia in the rabbit model: implications for sling surgery. J Urol, 171: 1970, 2004.
22. Bazi, T. M., Hamade, R. F., Abdallah Hajj Hussein, I. et al.: Polypropylene midurethral tapes do not have similar biologic and biomechanical performance in the rat. Eur Urol, 51: 1364, 2007.
23. UPDATE on Serious Complications Associated with Transvaginal Placement of Surgical Mesh for Pelvic Organ Prolapse: FDA Safety Communication, vol. 2015, 2011.
24. Mangera, A., Bullock, A. J., Chapple, C. R. et al.: Are biomechanical properties predictive of the success of prostheses used in stress urinary incontinence and pelvic organ prolapse? A systematic review. Neurourol Urodyn, 31: 13, 2012.
25. Webster, T. M., Gerridzen, R. G.: Gone in 24 hours: the feasibility of performing pubovaginal sling surgery with an overnight hospital stay. Can J Urol, 10: 1905, 2003.
26. Khan, Z. A., Nambiar, A., Morley, R. et al.: Long-term follow-up of a multicenter randomized controlled trial comparing tension-free vaginal tape, xenograft and autologous fascial slings for the treatment of stress urinary incontinence in women. BJU Int, 115: 968, 2015.
27. Gomelsky A, D. R.: Autograft, allograft, and xenograft slings: How are they different? Contemporary Urology, 17: 51, 2005.
28. Kaufman, M. R.: Contemporary role of autologous fascial bladder neck slings: a urology perspective. Urol Clin North Am, 39: 317, 2012.
29. Karlovsky, M. E., Kushner, L., Badlani, G. H.: Synthetic biomaterials for pelvic floor reconstruction. Curr Urol Rep, 6: 376, 2005.
30. Karlovsky, M. E., Thakre, A. A., Rastinehad, A. et al.: Biomaterials for pelvic floor reconstruction. Urology, 66: 469, 2005.
31. Dmochowski, R. R., Blaivas, J. M., Gormley, E. A. et al.: Update of AUA guideline on the surgical management of female stress urinary incontinence. J Urol, 183: 1906, 2010.
32. Godwin, J. W., Pinto, A. R., Rosenthal, N. A.: Macrophages are required for adult salamander limb regeneration. Proc Natl Acad Sci USA, 110: 9415, 2013.
33. Ogle, M. E., Segar, C. E., Sridhar, S. et al.: Monocytes and macrophages in tissue repair: Implications for immunoregenerative biomaterial design. Exp Biol Med (Maywood), 241: 1084, 2016.
34. Jetten, N., Verbruggen, S., Gijbels, M. J. et al.: Anti-inflammatory M2, but not pro inflammatory M1 macrophages promote angiogenesis in vivo. Angiogenesis, 17: 109, 2014.
35. Zajac, E., Schweighofer, B., Kupriyanova, T. A. et al.: Angiogenic capacity of M1- and M2 polarized macrophages is determined by the levels of TIMP-1 complexed with their secreted proMMP-9. Blood, 122: 4054, 2013.
36. Tattersall, I. W., Du, J., Cong, Z. et al.: In vitro modeling of endothelial interaction with macrophages and pericytes demonstrates Notch signaling function in the vascular microenvironment. Angiogenesis, 19: 201, 2016.
37. Loegl, J., Hiden, U., Nussbaumer, E. et al.: Hofbauer cells of M2a, M2b and M2c polarization may regulate feto-placental angiogenesis. Reproduction, 152: 447, 2016.
38. Ingman, W. V., Wyckoff, J., Gouon-Evans, V. et al.: Macrophages promote collagen fibrillogenesis around terminal end buds of the developing mammary gland. Dev Dyn, 235: 3222, 2006.
39. Lolmede, K., Campana, L., Vezzoli, M. et al.: Inflammatory and alternatively activated human macrophages attract vessel-associated stem cells, relying on separate HMGB1- and MMP-9-dependent pathways. J Leukoc Biol, 85: 779, 2009.
40. Spiller, K. L., Anfang, R. R., Spiller, K. J. et al.: The role of macrophage phenotype in vascularization of tissue engineering scaffolds. Biomaterials, 35: 4477, 2014.
41. Date, D., Das, R., Narla, G. et al.: Kruppel-like transcription factor 6 regulates inflammatory macrophage polarization. J Biol Chem, 289: 10318, 2014.
42. Xiong, Y., Lingrel, J. B., Wuthrich, M. et al.: Transcription Factor KLF2 in Dendritic Cells Downregulates Th2 Programming via the HIF-1alphagagged2/Notch Axis. MBio, 7, 2016.
43. Cora, M. C., King, D., Betz, L. J. et al.: Artifactual changes in Sprague-Dawley rat hematologic parameters after storage of samples at 3 degrees C. and 21 degrees C. J Am Assoc Lab Anim Sci, 51: 616, 2012.
44. Mia, S., Warnecke, A., Zhang, X. M. et al.: An optimized protocol for human M2 macrophages using M-CSF and IL-4/IL-10/TGF-beta yields a dominant immunosuppressive phenotype. Scand J Immunol, 79: 305, 2014.
45. Sridharan, R., Cameron, A. R., Kelly, D. J. et al.: Biomaterial based modulation of macrophage polarization: a review and suggested design principles. Materials Today, 18: 313, 2015.
46. Brown, B. N., Ratner, B. D., Goodman, S. B. et al.: Macrophage polarization: an opportunity for improved outcomes in biomaterials and regenerative medicine. Biomaterials, 33: 3792, 2012.
47. Rybalko, V., Hsieh, P. L., Merscham-Banda, M. et al.: The Development of Macrophage Mediated Cell Therapy to Improve Skeletal Muscle Function after Injury. PLoS One, 10: e0145550, 2015.
48. Koo, H. J., Lim, K. H., Jung, H. J. et al.: Anti-inflammatory evaluation of gardenia extract, geniposide and genipin. J Ethnopharmacol, 103: 496, 2006.
49. Koo, H. J., Song, Y. S., Kim, H. J. et al.: Antiinflammatory effects of genipin, an active principle of gardenia. Eur J Pharmacol, 495: 201, 2004.
50. Yu, S. X., Du, C. T., Chen, W. et al.: Genipin inhibits NLRP3 and NLRC4 inflammasome activation via autophagy suppression. Sci Rep, 5: 17935, 2015.
51. Uquillas, J. A., Akkus, O.: Modeling the electromobility of type-I collagen molecules in the electrochemical fabrication of dense and aligned tissue constructs. Annals of biomedical engineering, 40: 1641, 2012.
52. Cheng, X., Gurkan, U. A., Dehen, C. J. et al.: An electrochemical fabrication process for the assembly of anisotropically oriented collagen bundles. Biomaterials, 29: 3278, 2008.
53. Uquillas, J. A., Kishore, V., Akkus, O.: Effects of phosphate-buffered saline concentration and incubation time on the mechanical and structural properties of electrochemically aligned collagen threads. Biomed Mater, 6: 035008, 2011.
54. Younesi, M., Islam, A., Kishore, V. et al.: Fabrication of compositionally and topographically complex robust tissue forms by 3D-electrochemical compaction of collagen. Biofabrication, 7: 035001, 2015.
55. Kishore, V., Uquillas, J. A., Dubikovsky, A. et al.: In vivo response to electrochemically aligned collagen bioscaffolds. J Biomed Mater Res B Appl Biomater, 100: 400, 2012.
56. GD, L., PE, M., DM, K. et al.: Woven Collagen Biotextiles for Rotator Cuff Tendon Repair—An In Vivo Pilot Investigation. In: Transactions of the Annual Meeting of the Society For Biomaterials. Minneapolis, Minn., 2017.
57. Tsai, W. H., Yang, C. C., Li, P. C. et al.: Therapeutic potential of traditional Chinese medicine on inflammatory diseases. J Tradit Complement Med, 3: 142, 2013.
58. Younesi, M., Donmez, B. O., Islam, A. et al.: Corrigendum to “Heparinized collagen sutures for sustained delivery of PDGF-BB: Delivery profile, effects on tendon-derived cells In-Vitro” [Acta Biomater. 41 (2016) 100-109]. Acta Biomater, 51: 537, 2017.
59. Younesi, M., Donmez, B. O., Islam, A. et al.: Heparinized collagen sutures for sustained delivery of PDGF-BB: Delivery profile and effects on tendon-derived cells In-Vitro. Acta Biomater, 41: 100, 2016.
60. Mousa Younesi, A. I., Vipuil Kishore, James, M. Anderson, Ozan Akkus: Tenogenic Induction of Human MSCs by Anisotropically Aligned Collagen Biotextiles. Journal of Advanced Functional Materials, 2014.
61. Alfredo Uquillas, J., Kishore, V., Akkus, O.: Genipin crosslinking elevates the strength of electrochemically aligned collagen to the level of tendons. J Mech Behav Biomed Mater, 15: 176, 2012.
62. Hijaz, A., Bena, J., Daneshgari, F.: LONG-TERM EFFICACY OF A VAGINAL SLING PROCEDURE IN A RAT MODEL OF STRESS URINARY INCONTINENCE. The Journal of Urology, 173: 1817, 2005.
63. Hijaz, A., Daneshgari, F., Cannon, T. et al.: EFFICACY OF A VAGINAL SLING PROCEDURE IN A RAT MODEL OF STRESS URINARY INCONTINENCE. The Journal of Urology, 172: 2065, 2004.
64. Hijaz, A., Daneshgari, F., Sievert, K. D. et al.: Animal models of female stress urinary incontinence. J Urol, 179: 2103, 2008.
65. Zou, X. H., Zhi, Y. L., Chen, X. et al.: Mesenchymal stem cell seeded knitted silk sling for the treatment of stress urinary incontinence. Biomaterials, 31: 4872, 2010.
66. Hijaz, A., Bena, J., Daneshgari, F.: Long-term efficacy of a vaginal sling procedure in a rat model of stress urinary incontinence. J Urol, 173: 1817, 2005.
67. Kim, S. O., Na, H. S., Kwon, D. et al.: Bone-marrow-derived mesenchymal stem cell transplantation enhances closing pressure and leak point pressure in a female urinary incontinence rat model. Urol Int, 86: 110, 2011.
68. Chen, Y. M., Shen, R. W., Zhang, B. et al.: Regional tissue immune responses after sciatic nerve injury in rats. Int J Clin Exp Med, 8: 13408, 2015.
69. Badra, S., Andersson, K. E., Dean, A. et al.: A nonhuman primate model of stable urinary sphincter deficiency. J Urol, 189: 1967, 2013.
70. Zorn, K. C., Spiess, P. E., Singh, G. et al.: Long-term tensile properties of tension-free vaginal tape, suprapubic arc sling system and urethral sling in an in vivo rat model. J Urol, 177: 1195, 2007.
71. Badylak, S., Kokini, K., Tullius, B. et al.: Morphologic study of small intestinal submucosa as a body wall repair device. J Surg Res, 103: 190, 2002.
72. Peng, C. W., et al., External urethral sphincter activity in a rat model of pudendal nerve injury. Neurourol Urodyn, 2006. 25(4): p. 388-96.
73. Damaser, M. S., et al., Functional and neuroanatomical effects of vaginal distention and pudendal nerve crush in the female rat. J Urol, 2003. 170(3): p. 1027-31.
74. Chapin K, Khalifa A, Mbimba T, McClellan P, Anderson J, Novitsky Y, Hijaz A, Akkus O (2019) In vivo biocompatibility and time-dependent changes in mechanical properties of woven collagen meshes: A comparison to xenograft and synthetic mid-urethral sling materials. Journal of biomedical materials research Part B, Applied biomaterials 107 (3):479-489. doi:10.1002/jbm.b.34138.
75. Mariani E, Lisignoli G, Borzi R M, Pulsatelli L, Biomaterials: Foreign Bodies or Tuners for the Immune Response?, Int J Mol Sci 20(3) (2019) 636.
76. Boehler R M, Graham J G, Shea L D, Tissue engineering tools for modulation of the immune response, Biotechniques 51(4) (2011) 239-passim.
77. Sheikh Z, Brooks P J, Barzilay O, Fine N, Glogauer M, Macrophages, Foreign Body Giant Cells and Their Response to Implantable Biomaterials, Materials (Basel) 8(9) (2015) 5671-5701.
78. Hume D A, The mononuclear phagocyte system, Current opinion in immunology 18(1) (2006) 49-53.
79. Orecchioni M, Ghosheh Y, Pramod A B, Ley K, Macrophage Polarization: Different Gene Signatures in M1(LPS+) vs. Classically and M2(LPS-) vs. Alternatively Activated Macrophages, Frontiers in Immunology 10 (2019) 1084.
80. Murray P J, Macrophage Polarization, Annual Review of Physiology 79(1) (2017) 541-566.
81. Tugal D, Liao X, Jain M K, Transcriptional control of macrophage polarization, Arteriosclerosis, thrombosis, and vascular biology 33(6) (2013) 1135-44.
82. Wang N, Liang H, Zen K, Molecular mechanisms that influence the macrophage m1-m2 polarization balance, Frontiers in immunology 5 (2014) 614.
83. Lech M, Anders H J, Macrophages and fibrosis: How resident and infiltrating mononuclear phagocytes orchestrate all phases of tissue injury and repair, Biochimica et biophysica acta 1832(7) (2013) 989-97.
84. Rybalko V, Hsieh P L, Merscham-Banda M, Suggs L J, Farrar R P, The Development of Macrophage-Mediated Cell Therapy to Improve Skeletal Muscle Function after Injury, PLoS One 10(12) (2015) e0145550.
85. Koci Z, Sridharan R, Hibbitts A J, Kneafsey S L, Kearney C J, O'Brien F J, The Use of Genipin as an Effective, Biocompatible, Anti-Inflammatory Cross-Linking Method for Nerve Guidance Conduits, Advanced biosystems 4(3) (2020) e1900212.
86. McWhorter F Y, Wang T, Nguyen P, Chung T, Liu W F, Modulation of macrophage phenotype by cell shape, Proc. Natl. Acad. Sci. U.S.A 110(43) (2013) 17253-17258.
87. Shindo S, Hosokawa Y, Hosokawa I, Ozaki K, Matsuo T, Genipin inhibits IL-1beta-induced CCL20 and IL-6 production from human periodontal ligament cells, Cellular physiology and biochemistry: international journal of experimental cellular physiology, biochemistry, and pharmacology 33(2) (2014) 357-64.
88. Minami K, Hiwatashi K, Ueno S, Sakoda M, lino S, Okumura H, Hashiguchi M, Kawasaki K, Kurahara H, Mataki Y, Maemura K, Shinchi H, Natsugoe S, Prognostic significance of CD68, CD163 and Folate receptor-β positive macrophages in hepatocellular carcinoma, Exp Ther Med 15(5) (2018) 4465-4476.
89. Higashi-Kuwata N, Jinnin M, Makino T, Fukushima S, Inoue Y, Muchemwa F C, Yonemura Y, Komohara Y, Takeya M, Mitsuya H, Ihn H, Characterization of monocyte/macrophage subsets in the skin and peripheral blood derived from patients with systemic sclerosis, Arthritis Research & Therapy 12(4) (2010) R128.
90. Learn G D, McClellan P E, Knapik D M, Cumsky J L, Webster-Wood V, Anderson J M, Gillespie R J, Akkus O, Woven collagen biotextiles enable mechanically functional rotator cuff tendon regeneration during repair of segmental tendon defects in vivo, Journal of biomedical materials research. Part B, Applied biomaterials 107(6) (2019) 1864-1876.
91. Sapudom J, Mohamed W K E, Garcia-Sabate A, Alatoom A, Karaman S, Mahtani N, Teo J C, Collagen Fibril Density Modulates Macrophage Activation and Cellular Functions during Tissue Repair, Bioengineering (Basel, Switzerland) 7(2) (2020).
92. Zhang F, Parayath N N, Ene C I, Stephan S B, Koehne A L, Coon M E, Holland E C, Stephan M T, Genetic programming of macrophages to perform anti-tumor functions using targeted mRNA nanocarriers, Nature communications 10(1) (2019) 3974.
93. Fleetwood A J, Lawrence T, Hamilton J A, Cook A D, Granulocyte-macrophage colony-stimulating factor (CSF) and macrophage CSF-dependent macrophage phenotypes display differences in cytokine profiles and transcription factor activities: implications for CSF blockade in inflammation, Journal of immunology (Baltimore, Md.: 1950) 178(8) (2007) 5245-52.
94. Van den Bossche J, Baardman J, Otto N A, van der Velden S, Neele A E, van den Berg S M, Luque-Martin R, Chen H J, Boshuizen M C, Ahmed M, Hoeksema M A, de Vos A F, de Winther M P, Mitochondrial Dysfunction Prevents Repolarization of Inflammatory Macrophages, Cell reports 17(3) (2016) 684-696.
95. Manickam B, Sreedharan R, Elumalai M, ‘Genipin’—the natural water soluble cross-linking agent and its importance in the modified drug delivery systems: an overview, Current drug delivery 11(1) (2014) 139-45.
96. Wang Q-S, Xiang Y, Cui Y-L, Lin K-M, Zhang X-F, Dietary blue pigments derived from genipin, attenuate inflammation by inhibiting LPS-induced iNOS and COX-2 expression via the NF-κB inactivation, PLoS One 7(3) (2012) e34122-e34122.
97. Lively S, Schlichter L C, Microglia Responses to Pro-inflammatory Stimuli (LPS, IFNγ+TNFα) and Reprogramming by Resolving Cytokines (IL-4, IL-10), Front Cell Neurosci 12 (2018) 215-215.
98. Wang Y, Bao J, Wu X, Wu Q, Li Y, Zhou Y, Li L, Bu H, Genipin crosslinking reduced the immunogenicity of xenogeneic decellularized porcine whole-liver matrices through regulation of immune cell proliferation and polarization, Sci Rep 6 (2016) 24779-24779.
99. Sridharan R, Ryan E J, Kearney C J, Kelly D J, O'Brien F J, Macrophage Polarization in Response to Collagen Scaffold Stiffness Is Dependent on Cross-Linking Agent Used To Modulate the Stiffness, ACS Biomaterials Science & Engineering 5(2) (2019) 544-552.
100. Hwa J S, Mun L, Kim H J, Seo H G, Lee J H, Kwak J H, Lee D-U, Chang K C, Genipin Selectively Inhibits TNF-α-activated VCAM-1 But Not ICAM-1 Expression by Upregulation of PPAR-γ in Human Endothelial Cells, Korean J Physiol Pharmacol 15(3) (2011) 157-162.
101. Biswas S K, Mantovani A, Macrophage plasticity and interaction with lymphocyte subsets: cancer as a paradigm, Nature immunology 11(10) (2010) 889-96.
102. Kelly-Welch A E, Hanson E M, Boothby M R, Keegan A D, Interleukin-4 and interleukin-13 signaling connections maps, Science 300(5625) (2003) 1527-8.
103. Martinez F O, Helming L, Gordon S, Alternative activation of macrophages: an immunologic functional perspective, Annual review of immunology 27 (2009) 451-83.
104. Szanto A, Balint B L, Nagy Z S, Barta E, Derso B, Pap A, Szeles L, Poliska S, Oros M, Evans R M, Barak Y, Schwabe J, Nagy L, STAT6 transcription factor is a facilitator of the nuclear receptor PPARγ-regulated gene expression in macrophages and dendritic cells, Immunity 33(5) (2010) 699-712.
105. Odegaard J I, Ricardo-Gonzalez R R, Goforth M H, Morel C R, Subramanian V, Mukundan L, Red Eagle A, Vats D, Brombacher F, Ferrante A W, Chawla A, Macrophage-specific PPARgamma controls alternative activation and improves insulin resistance, Nature 447(7148) (2007) 1116-20.
106. Das A, Sinha M, Datta S, Abas M, Chaffee S, Sen C K, Roy S, Monocyte and macrophage plasticity in tissue repair and regeneration, Am J Pathol 185(10) (2015) 2596-2606.
107. Rath M, Muller I, Kropf P, Closs E I, Munder M, Metabolism via Arginase or Nitric Oxide Synthase: Two Competing Arginine Pathways in Macrophages, Frontiers in immunology 5 (2014) 532-532.
108. El Kasmi K C, Qualls J E, Pesce J T, Smith A M, Thompson R W, Henao-Tamayo M, Basaraba R J, Konig T, Schleicher U, Koo M S, Kaplan G, Fitzgerald K A, Tuomanen E I, Orme I M, Kanneganti T D, Bogdan C, Wynn T A, Murray P J, Toll-like receptor-induced arginase 1 in macrophages thwarts effective immunity against intracellular pathogens, Nature immunology 9(12) (2008) 1399-406.
109. Rutschman R, Lang R, Hesse M, Ihle J N, Wynn T A, Murray P J, Cutting edge: Stat6-dependent substrate depletion regulates nitric oxide production, Journal of immunology (Baltimore, Md.: 1950) 166(4) (2001) 2173-7.
110. Hesse M, Modolell M, La Flamme A C, Schito M, Fuentes J M, Cheever A W, Pearce E J, Wynn T A, Differential regulation of nitric oxide synthase-2 and arginase-1 by type 1/type 2 cytokines in vivo: granulomatous pathology is shaped by the pattern of L-arginine metabolism, Journal of immunology (Baltimore, Md.: 1950) 167(11) (2001) 6533-44.
111. Numata K, How to define and study structural proteins as biopolymer materials, Polymer Journal 52 (2020) 1043-1056.
112. Zhang B, Xu L, Ding K, Dyeing/cross-linking property of natural iridoids to protein fibers: Part I. Preparation of four natural iridoids and their dyeing/cross-linking (tanning) property to hide powder, JALCA 106 (2011) 121-126.

What is claimed is:

1. A biologic material comprising:
(a) a crosslinked structural protein; and
(b) macrophages seeded on the crosslinked structural protein.

2. The biologic material of claim 1, wherein the crosslinked structural protein comprises one or more of a crosslinked collagen, a crosslinked gelatin, a crosslinked elastin, or a crosslinked keratin.

3. The biologic material of claim 2, wherein the crosslinked structural protein comprises a crosslinked collagen.

4. The biologic material of claim 1, wherein the crosslinked structural protein has been crosslinked with an iridoid crosslinking agent.

5. The biologic material of claim 4, wherein the crosslinked structural protein has been crosslinked with one or more of genipin, loganin aglycone, oleuropein aglycone, or E-6-O-methoxycinnamoyl scandoside methyl ester aglycone.

6. The biologic material of claim 1, wherein the crosslinked structural-protein forms a crosslinked structural-protein mesh.

7. The biologic material of claim 6, wherein the crosslinked structural-protein mesh comprises a crosslinked collagen mesh.

8. The biologic material of claim 7, wherein the crosslinked collagen mesh comprises a crosslinked woven collagen mesh.

9. The biologic material of claim 1, wherein the crosslinked structural-protein forms a crosslinked structural-protein gel.

10. The biologic material of claim 9, wherein the crosslinked structural-protein gel comprises a crosslinked collagen gel.

11. The biologic material of claim 1, wherein the macrophages comprise autologous-blood derived macrophages and/or allogeneic-blood derived macrophages.

12. The biologic material of claim 1, wherein the macrophages comprise M2 macrophages.

13. The biologic material of claim 1, wherein the macrophages have been seeded on the crosslinked structural protein at a seeding density of 5,000 to 100,000 macrophages/mm³ of the biologic material.

14. The biologic material of claim 1, wherein to the extent that human or animal cells other than macrophages are present on the crosslinked structural protein of the biologic material, more macrophages are present than the other human or animal cells.

15. The biologic material of claim 1, wherein to the extent that human or animal cells other than macrophages are attached to the crosslinked structural protein of the biologic material, more macrophages are attached than the other human or animal cells.

16. A method of use of the biologic material of claim 1 for an immunoregenerative treatment in a patient in need thereof comprising steps of:
(1) seeding the macrophages on the crosslinked structural protein, thereby obtaining the biologic material; and
(2) implanting the biologic material into the patient.

17. The method of claim 16, wherein the macrophages comprise M2 macrophages derived from monocytes, the method further comprising steps of:
(0.1) treating the monocytes with Macrophage Colony Stimulating Factor to obtain M0 macrophages; and
(0.2) treating the M0 macrophages with a mixture of TGF-β, IL-4, IL-10, and/or IL-13 to obtain the M2 macrophages.

18. The method of claim 16, wherein the macrophages comprise M2 macrophages derived from M0 macrophages, the method further comprising a step (0.3) of treating the M0 macrophages with genipin in a solution to obtain the M2 macrophages.

19. The method of claim 16, further comprising treating the macrophages with IL-4 during step (1).

20. The method of claim 16, wherein the immunoregenerative treatment comprises one or more of treatment of stress urinary incontinence, treatment of pelvic organ prolapse, hernia repair, or an orthopedic application.
